(19) United States
(12) Patent Application Publication
(10) Pub. No.: US 2002/0115173 A1
(43) Pub. Date: Aug. 22, 2002

(54) Short peptides from the 'A-region' of protein kinases which selectively modulate protein kinase activity

(75) Inventor: Shmuel A. Ben-Sasson, Jerusalem (IL)

Correspondence Address: BROWDY AND NEIMARK, P.L.L.C., 624 NINTH STREET, NW, SUITE 300, WASHINGTON, DC, 20001-5303, US

(73) Assignee: Children's Medical Center Corporation

(21) Appl. No.: 09/734,520

(22) Filed: Dec. 11, 2000

(51) Int. Cl. C12N 9/12; C12P 21/04; G01N 33/537

(52) U.S. Cl. 435/194; 435/70.21; 435/7.92

(57) ABSTRACT

Peptides which are peptide derivatives of the A region of a protein kinase can modulate the activity of protein kinases. The activity of a protein kinase in a subject can be modulated by administering one or more of these peptides.

### BACKGROUND OF THE INVENTION

There are a group of proteins that constitute the eukaryotic protein kinase superfamily. Enzymes of this class specifically phosphorylate serine, threonine or tyrosine residues of intracellular proteins. These enzymes are important in mediating signal transduction in multicellular organisms. Many of the protein kinases are part of transmembrane receptors. Others occur as intracellular proteins which take part in signal transduction within the cell, including signal transduction to the nucleus and activation of other proteins. Other protein kinases, such as G protein-coupled receptor kinases, attenuate transmembrane signaling.

As such, phosphorylation of serine, threonine or tyrosine by protein kinases is an important mechanism for regulating intracellular events in response to environmental changes. A wide variety of cellular events are regulated by protein kinases. A few examples include cellular proliferation, cellular differentiation, the ability of cells to enter and/or complete mitosis, cellular transformation by RNA viruses, oncogenesis, immune responses, inflammatory responses and the control of carbohydrate or fat metabolism.

Enhanced protein kinase activity can lead to persistent stimulation by secreted growth factors and other growth inducing factors which, in turn, can lead to proliferative diseases such as cancer, to nonmalignant proliferative diseases such as arteriosclerosis, psoriasis and to inflammatory response such as septic shock. Decreased function can also lead to disease. For example, a decrease in the activity of insulin receptor kinase is a cause of various types of diabetes. Severe reduction of the B cell progenitor kinase leads to human X-linked agammaglobulinemia.

Thus, agents which can modulate (increase or decrease) the activity of protein kinases have great potential for the treatment of a wide variety of diseases and conditions such as cancer, obesity, autoimmune disorders, inflammation and diabetes. Such agents also have utility in deciphering the mode of action of protein kinases and how these proteins regulate cellular functions and activities.

### SUMMARY OF THE INVENTION

It has now been found that short peptides which are derivatives of the aC to b4 region of a protein kinase (hereby A region) can significantly affect the activities of cells expressing the protein kinase when incubated with the cells (the “A region” is defined hereinbelow). Based on the aforementioned discoveries, novel peptides are disclosed herein which are peptide derivatives of the A region of protein kinases. Also disclosed are methods of identifying a peptide derivative of an A region of a protein kinase that modulates the activity of the protein kinase. Methods of modulating the activity of a protein kinase in a subject are also disclosed.

One embodiment of the present invention is a novel peptide which is a peptide derivative of the A region of a protein kinase. The peptide comprises between about five and about twenty amino acid residues or amino acid residue analogs of the A region. The peptide modulates the activity of the protein kinase. The N-terminus and/or C-terminus of the peptide can be substituted or unsubstituted. The peptide can be linear or cyclic.

Another embodiment of the present invention is a method of modulating the activity of a protein kinase in a subject. The method comprises administering a therapeutically effective amount of a peptide that is a derivative of the A region of the protein kinase, as described above.

Yet another embodiment of the present invention is a method of identifying a peptide which modulates the activity of a protein kinase. The method comprises providing a “test peptide” which has from about five to about twenty amino acids or amino acid analogs and which is a peptide derivative of the A region of the protein kinase. The test peptide is incubated with cells having a cellular activity or function under the control of the protein kinase under conditions suitable for assessing the activity of the protein kinase. The activity of the protein kinase is assessed and compared with the activity of the protein kinase in cells of the same cell type grown under the same conditions in the absence of the test peptide. A greater or lesser activity compared with cells grown in the absence of the test peptide indicates that the test peptide modulates the activity of the protein kinase.

The peptides of the present invention can be used in the treatment of a wide variety of diseases caused by overactivity or underactivity of a protein kinase. Examples include, but are not limited to, favorable tissue remodeling such as bone formation, reduced scar formation, enhanced hair growth, induction of differentiation of pancreatic duct cells, inhibition of the growth of adipose tissue, cancer treatment, diseases caused by proliferation of smooth muscle (e.g. restenosis and atherosclerosis), skin disorders, diabetes, obesity, diseases of the central nervous system, inflammatory disorders, autoimmune diseases and other immune disorders, osteoporosis and cardiovascular diseases. The peptides of the present invention also have in vitro utilities, for example, in the generation of antibodies that specifically bind the protein kinase from which the peptide was derived. These antibodies can be used to identify cells expressing the protein kinase and to study the intracellular distribution of the protein kinase. In addition, the peptides of the present invention can be used to identify and quantitate ligands that bind the A region of the protein kinase from which the peptide was derived.

### BRIEF DESCRIPTION OF THE DRAWINGS

FIGS. 1A-1B are a table illustrating the amino acid sequences of the A region of the following protein kinases: Src, Yes, Fyn, Fgr, Lyn, Hck, Lck (SEQ ID NO. 1 to 7); Csk and Matk (SEQ ID NO. 8 to 9); focal adhesion kinase (FAK) (SEQ ID NO. 10); c-Abl (SEQ ID NO. 11); endothelial growth factor receptors Tie, Tek, FGF receptor (Bek, Flg, FGFR3, FGFR4), PDGF receptor a and b, Flt 1 and 4 and Flk1 (SEQ ID NO. 12 to 19); HGF receptors c-Met, c-Sea and Ron (SEQ ID NO. 20 to 22); EGF receptor (EGFR, ErbB2, ErbB3, ErbB4) (SEQ ID NO. 23 to 26); Ret (SEQ ID NO. 27); NGF receptors (Trk) (SEQ ID NO. 28 to 29); Syk and Zap70 (SEQ ID NO. 30 to 31); Jak kinases 1 through 3 and Tyk2 (SEQ ID NO. 32 to 35); insulin receptor kinase (IRK) (SEQ ID NO. 36); Activin receptor-like kinases 1 through 6 (ALK1, 2, 3, 4, 5, 6) (SEQ ID NO. 37 to 40); discoidin domain receptors 1 and 2 (DDR) (SEQ ID NO. 41 to 42); ACK (SEQ ID NO. 43); Ephrin receptor B4 (SEQ ID NO. 44); TEC (SEQ ID NO. 45); Polo family kinases Plk, Plx1, polo, SNK, CDC5, Sak, Prk, Fnk and Plo1 (SEQ ID NO. 46 to 53).

FIGS. 2A-2E are a group of sequences illustrating the consensus amino acid sequences of the A region found among the family of protein kinases. Also shown are examples of conservative substitutions in these amino acid sequences. An “*” indicates an aliphatic, substituted aliphatic, benzylic, substituted benzylic, aromatic or substituted aromatic ester of glutamic acid or aspartic acid.

FIGS. 3A-3B are a Table illustrating the sequences of the following peptides: Plk K035A100; Plx1 K036A100; polo K037A100; SNK K038A100; CDC5 K039A100; Sak K040A100; Prk K041A100; Plo1 K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Abl K061A100; Tie K062A100; PDGFR-b K064A100; PDGFR-a K065A100; Flt1 K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K0; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A118 K094A119 K094A131 K094A132 K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively).

Peptides are N-myristylated and C-amidated. “K+” indicates a benzoylated lysine residue (epsilon amino). “C5” indicates a lysine-epsilon-amino cysteine. “C6” indicates an alanine-beta-amino cysteine. FIG. 3 shows that one or more glycine residues can be added to the N-terminus of the native A-region amino acid sequence. FIG. 3 also indicates from which protein kinase each peptide is derived.

FIG. 4 is a graphical representation of the percent change in glucose uptake of adipose tissue cells from rats. The treated cells were incubated with different concentrations of insulin or an IRK-derived peptide with/without insulin.

### DETAILED DESCRIPTION OF THE INVENTION

A protein kinase (hereinafter “PK”) is an intracellular or membrane bound protein which uses the gamma phosphate of ATP or GTP to generate phosphate monoesters on the hydroxyl group of a serine or threonine residue, or on the phenolic group of a tyrosine residue. PKs have homologous “kinase domains” or “catalytic domains” which carry out this phosphorylation. Based on a comparison of a large number of protein kinases, it is now known that the kinase domain of protein kinases can be divided into twelve subdomains. These are regions that are generally uninterrupted by large amino acid insertions and they contain characteristic patterns of conserved residues (Hanks and Hunter, “The Eukaryotic Protein Kinase Superfamily”, in Hardie and Hanks ed., The Protein Kinase Facts Book, Volume I, Academic Press, Chapter 2, 1995). These subdomains are referred to as Subdomain I through Subdomain XII.

Because of the high degree of homology found in the subdomains of different protein kinases, the amino acid sequences of the domains of different PKs can be aligned. Frequently, the alignment is with reference to the prototypical protein kinase PKA-Cα, as known in the art. Currently, the catalytic domains of a large number of protein kinases have been aligned and tables showing these alignments are available from various published sources, such as, for example, in the article by Hanks and Quinn in Methods of Enzymology 200, 38-62 (1991) or in the PKR Web Site: WWW.sdsc.edu/kinases.

The “A region” referred to herein is found within the kinase domain of PKs in Subdomain III and Subdomain IV. With respect to the amino acid sequence of the prototypical protein kinase PKA-Cα, the A region can be said to correspond to a contiguous sequence of about eighteen amino acid residues found between about amino acids 92 and 109 of PKA-Cα. Of course, in some PKs, extra amino acids can be present in this region and the size of the A region can, therefore, include more than 18 amino acids in length.

In terms of the three dimensional structure of PKs, the kinase domain of PKs has been found to contain at least nine alpha helices, referred to as helix A through helix I and nine beta sheets, referred to as b1 through b9 (Tabor et al., Phil. Trans. R. Soc. Lond. B340:315 (1993), Mohammadi et al., Cell 86:577 (1996) and Hubbard et al., Nature 372:746 (1994)). Relationships between the primary structure of a large number of protein kinases and their corresponding three dimensional structure is well known in the art. With respect to the three dimensional structure of protein kinases, the A region is a contiguous sequence of about five to twenty amino acids beginning at the middle of the αC helix (hereby aC) and ending at the beginning of the b4 beta sheet.

Optionally, the C-terminus or the N-terminus of the peptides of the present invention, or both, can be substituted with a carboxylic acid protecting group or an amine protecting group, respectively. Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the peptide into a cell, for example, by reducing the hydrophilicity and increasing the lipophilicity of the peptide. In addition, a modified lysine residue can be added to the carboxy terminus to enhance biological activity. Examples of the lysine modification include the addition of an aromatic substitute, such as benzoyl, or an aliphatic group, such as acyl, or a myristic or stearic acid, at the epsilon amino group of the lysine residue. Examples of N-terminal protecting groups include acyl groups (—CO—R₁) and alkoxy carbonyl or aryloxy carbonyl groups (—CO—O—R₁), wherein R₁ is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, phenyl-CO—, substituted phenyl-CO—, benzyl-CO— and (substituted benzyl)-CO—. Examples of alkoxy carbonyl and aryloxy carbonyl groups include CH₃—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. In order to facilitate the N-acylation, one to four glycine residues can be added to the N-terminus of the sequence. The carboxyl group at the C-terminus can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, -N(methyl)(ethyl), —NH(benzyl), —N(C1-C4 alkyl)(benzyl), —NH(phenyl), —N(C1-C4 alkyl)(phenyl), —OCH₃, —O—(ethyl), —O—(n-propyl), —O—(n-butyl), —O—(iso-propyl), —O—(sec-butyl), —O—(t-butyl), —O-benzyl and —O-phenyl.

The term “peptide derivative” has four meanings as the term is applied to the A region of a protein kinase. One of the meanings is a relatively short amino acid sequence that includes a peptide having the amino acid sequence of the A region. The term “peptide derivative” of the A region also means a subsequence of the A region of the PK. A subsequence of a protein region is a contiguous sequence of from about five to about twenty amino acids or conservatively substituted amino acids found within a larger sequence. Thus, a subsequence of the A region is a contiguous sequence of from about five to about twenty amino acids or conservatively substituted amino acids found within the A region. A subsequence of the A region can also be referred to as a “fragment” of the A region.

A “peptide derivative” also means a peptide having a “modified sequence” in which one or more amino acids in the original sequence or subsequence have been substituted with a naturally occurring amino acid, a conservatively substituted amino acid (also referred to as a “modified amino acid”) or a functional peptidomimetic. Finally, the term “peptide derivative” means a peptide in which one or more amino acids, conservatively substituted amino acids or functional peptidomimetics is (are) inserted as one or more spacer groups within the original sequence or subsequence of the peptide.

In one aspect of the present invention, the peptide derivative has the sequence of a subsequence of the A region of a PK, with the proviso that any one amino acid residue in the peptide derivative differs from the corresponding amino acid residue in the subsequence. For example, if the subsequence is [AA₁]-[AA₂]-AA₃]-[AA₄]-[AA₅], then the peptide derivative can be [AA₁′]-[AA₂]-[AA₃]-[AA₄]-[AA₅], [AA₁]-[AA₂′]-[AA₃]-[AA₄]-[AA₅], [AA₁]-[AA₂]-[AA₃′]-[AA₄]-[AA₅], [AA₁]-[AA₂]-[AA₃]-[AA₄′]-[AA₅] and [AA₁]-[AA₂]-AA₃]-[AA₄]-[AA₅′], wherein [AA′] is a naturally occurring or a modified amino acid different from [AA]. In another aspect of the present invention, the peptide derivative has a sequence corresponding to a subsequence of the A region of a PK, with the proviso that any two amino acid residues in the peptide derivative differ from the corresponding amino acid residues in the subsequence.

An “amino acid residue” is a moiety found within a peptide and is represented by —NH—CHR—CO—, wherein R is the side chain of a naturally occurring amino acid. When referring to a moiety found within a peptide, the terms “amino acid residue” and “amino acid” are used in this application. An “amino acid residue analog” is either a peptidomimetic or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. When referring to a moiety found within a peptide, the terms “amino acid residue analog” and “amino acid analog” are used interchangeably in this application. Amino acid analogs are well-known in the art; a large number of these analogs are commercially available.

A “conservatively substituted amino acid”, also called a “conservatively substituted amino acid residue”, is an amino acid analog which, when substituted for a native (original) amino acid of the A region peptide or is inserted as a spacer group in the amino acid sequence of the A region peptide, does not severely alter the modulating activity of the peptide. A peptidomimetic of the naturally occurring amino acid, as well documented in the literature known to the skilled practitioner, also referred to as a “functional peptidomimetic” is an organic moiety which, when substituted for a native (original) amino acid of the A region peptide or is inserted as a spacer group in the amino acid sequence of the A region peptide, also does not severely alter the modulating activity of the peptide. The ability of an A region peptide derivative to affect the activities of cells expressing the target protein kinases is not markedly different from the modulating ability of the native or original A region peptide either when a conservatively substituted amino acid or functional peptidomimetic replaces a native amino acid of the native or original A region peptide, or when a conservatively substituted amino acid or functional peptidomimetic is inserted in an amino acid sequence of an A region peptide.

As used herein, aliphatic groups are straight chained, branched or cyclic C1 -C8 hydrocarbons that are completely saturated, which contain one or two heteroatoms such as nitrogen, oxygen or sulfur and/or which contain one or more units of unsaturation. Aromatic groups are carbocyclic aromatic groups such as phenyl and naphthyl and heterocyclic aromatic groups such as imidazolyl, indolyl, thienyl, furanyl, pyridyl, pyranyl, pyrrolyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F), —O(aliphatic, substituted aliphatic, benzyl, substituted benzyl, aryl or substituted aryl group), —CN, —NO₂, —COOH, —NH_{2,} —NH(aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more of these substituents.

Suitable substitutions for amino acid residues in the sequence of an A region or a subsequence of an A region include conservative substitutions which result in peptide derivatives which modulate the activity of a PK. Among the categories of amino acid substitutions, conservative substitutions are preferred in this invention. Particularly, preferred, are amino acid substitutions where one, two or three amino acids are substituted by a conservative substitution. A “conservative substitution” is a substitution in which the substituting amino acid (naturally occurring or modified) has about the same steric and electronic properties as the amino acid being substituted. Thus, the substituting amino acid would have the same or a similar functional group in the side chain as the original amino acid.

A “conservative substitution” can also be achieved by utilizing a substituting amino acid that is identical to the amino acid being substituted except that a functional group in the side chain is coupled to a suitable protecting group. Suitable protecting groups are described in Green and Wuts, “Protecting Groups in Organic Synthesis”, John Wiley and Sons, Chapters 5 and 7, 1991, the teachings of which are incorporated herein by reference. As with N-terminal and C-terminal protecting groups, preferred protecting groups are those which facilitate transport of the peptide into a cell, for example, by reducing the hydrophilicity and increasing the lipophilicity of the peptide, and which can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester, more preferably as a benzyl ester.

Provided below are groups of naturally occurring and modified amino acids in which each amino acid in a group has similar electronic and steric properties. Thus, a conservative substitution is made by substituting an amino acid with another amino acid from the same group. It is to be understood that these groups are non-limiting, i.e. that there are additional modified amino acids which could be included in each group.

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably, Group I includes leucine, isoleucine, valine and methionine.

Group II includes glycine, alanine, valine, serine, cysteine, threonine and a modified amino acid having an ethyl side chain. Preferably, Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine, β-cycloarginine, g-hydroxyarginine, N-amidinocitruline and 2-amino-4-guanidinobutanoic acid, homologs of lysine, homologs of arginine and ornithine. Preferably, Group V includes histidine, lysine, arginine, and omithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

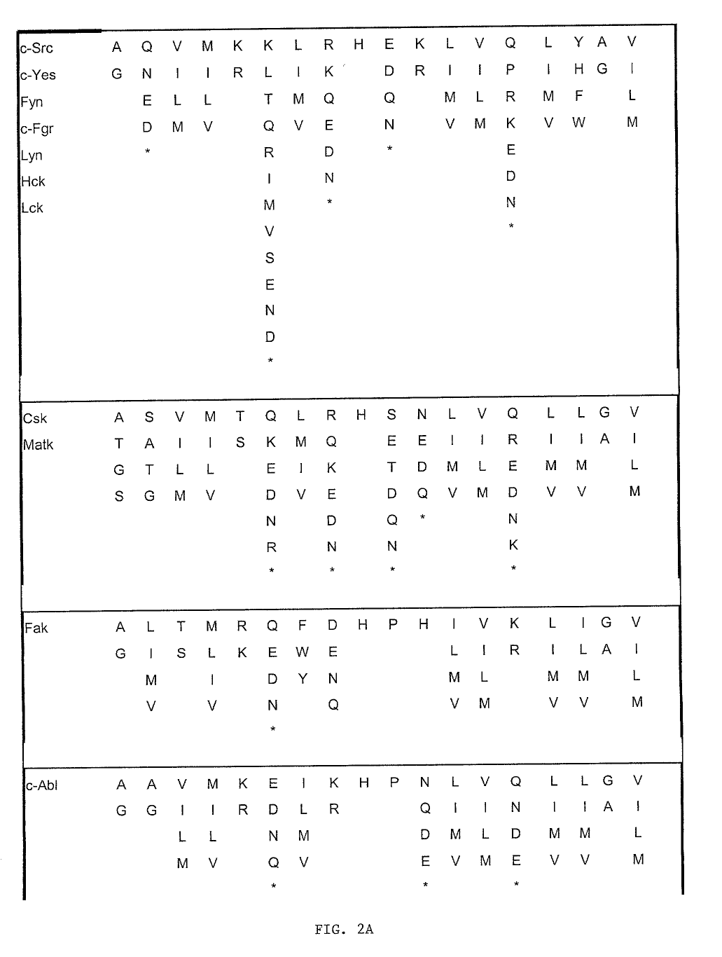

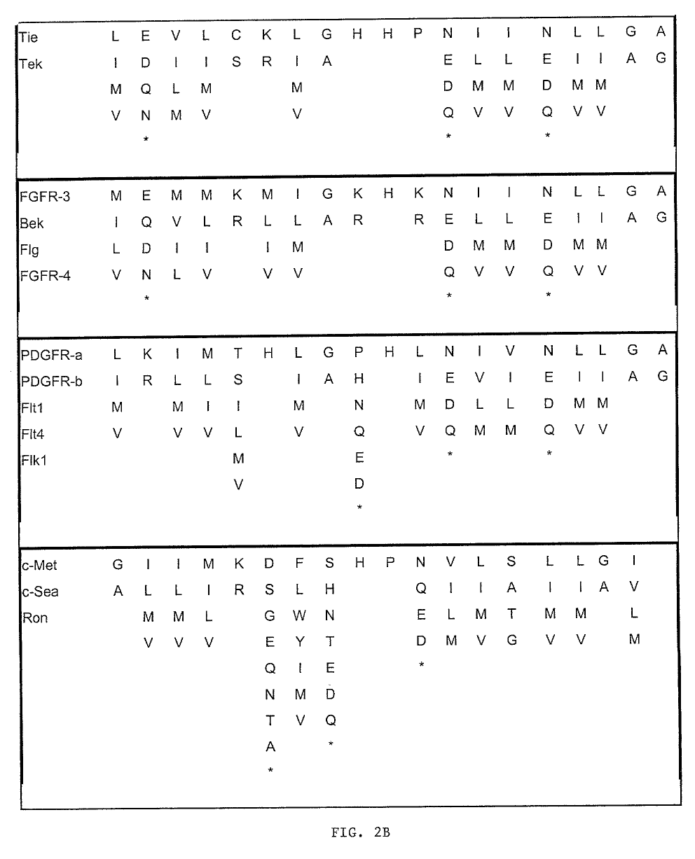

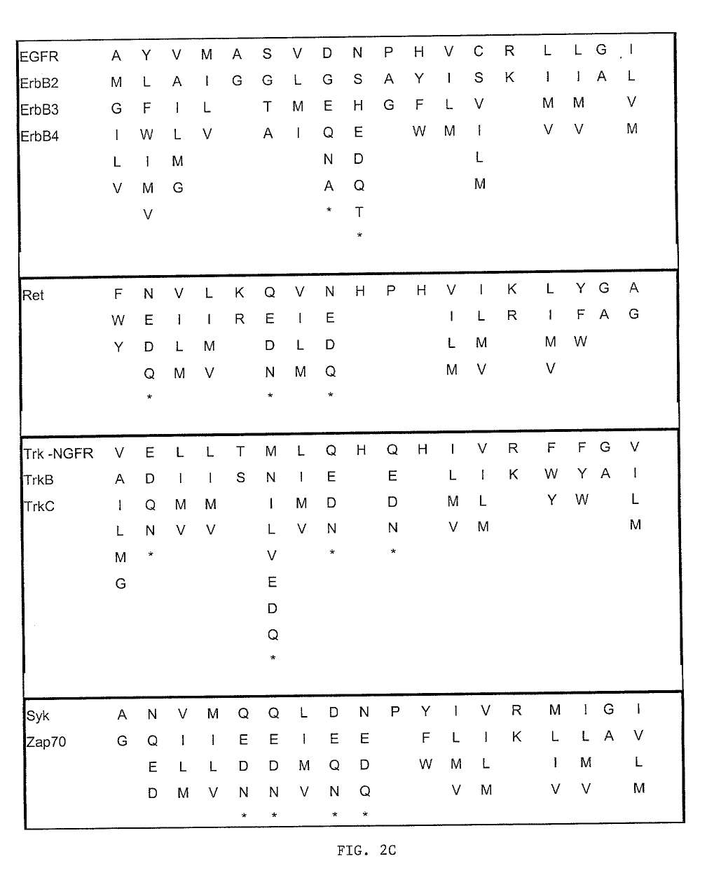

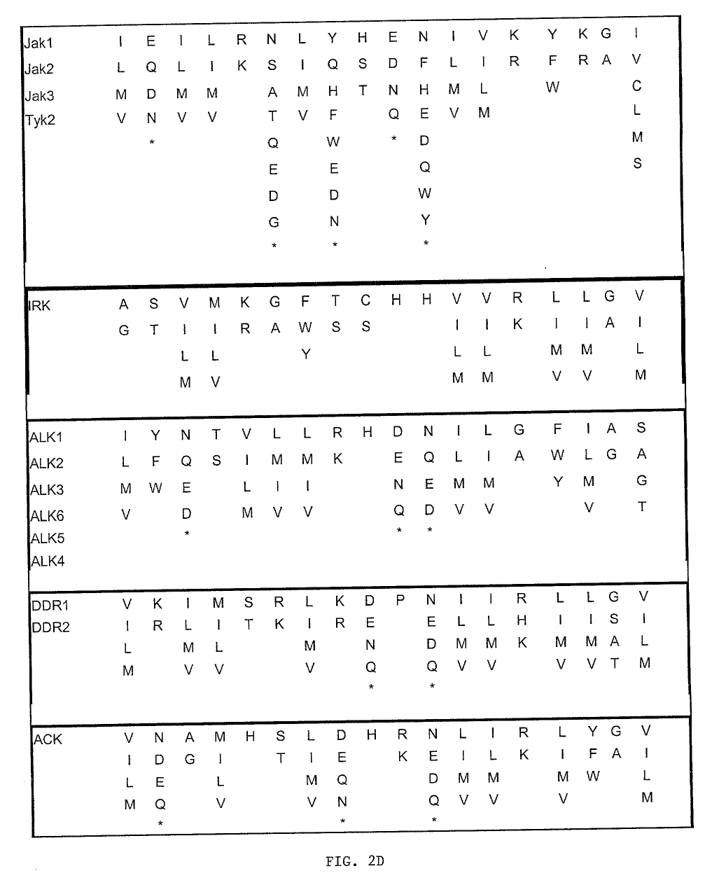

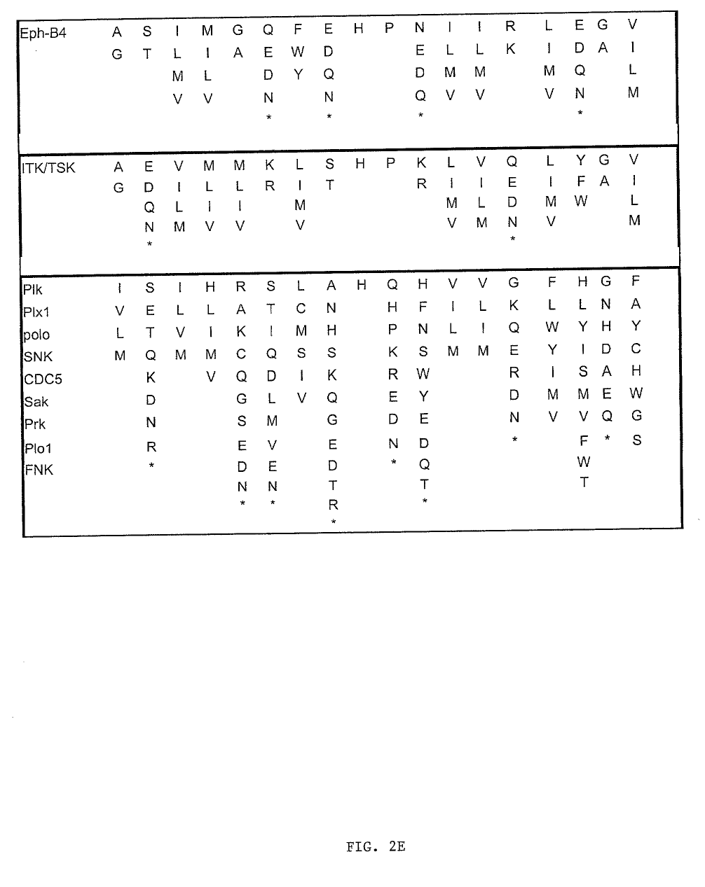

In another aspect, suitable substitutions for amino acid residues in the sequence of an A region or a subsequence of an A region include “severe substitutions” which result in peptide derivatives which modulate the activity of a PK. Severe substitutions which result in peptide derivatives that modulate the activity of a PK are much more likely to be possible in positions which are not highly conserved throughout the family of protein kinases than at positions which are highly conserved. FIG. 2 shows the consensus sequences of the five to twenty amino acids of the A region of PKs. Because D-amino acids have a hydrogen at a position identical to the glycine hydrogen side-chain, D-amino acids or their analogs can often be substituted for glycine residues.

A “severe substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cycohexylmethyl glycine for alanine, isoleucine for glycine, or —NH—CH[(—CH₂)₅—COOH]—CO— for aspartic acid. Alternatively, a functional group may be added to the side chain, deleted from the side chain or exchanged with another functional group. Examples of severe substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄COOH for the side chain of serine. These examples are not meant to be limiting.

“Peptidomimetics” can be substituted for amino acid residues in the peptides of this invention. These peptidomimetics either replace amino acid residues or act as spacer groups within the peptides. The peptidomimetics often have steric, electronic or configurational properties similar to the replaced amino acid residues but such similarities are not necessarily required. The only restriction on the use of peptidomimetics is that the peptides retain their protein kinase modulating activity. Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110, 5875-5880 (1988)); isosteres of amide bonds (Jones et al., Tetrahedron Lett. 29, 3853-3856 (1988)); LL-3-amino-2-propenidone-6-carboxylic acid (LL-Acp) (Kemp et al., J. Org. Chem. 50, 5834-5838 (1985)). Similar analogs are shown in Kemp et al., Tetrahedron Lett. 29, 5081-5082 (1988) as well as Kemp et al., Tetrahedron Lett. 29, 5057-5060 (1988), Kemp et al., Tetrahedron Lett. 29, 4935-4938 (1988) and Kemp et al., J. Org. Chem.54, 109-115 (1987). Other suitable peptidomimetics are shown in Nagai and Sato, Tetrahedron Lett. 26, 647-650 (1985); Di Maio et al., J. Chem. Soc. Perkin Trans., 1687 (1985); Kahn et al., Tetrahedron Lett. 30, 2317 (1989); Olson et al., J. Am. Chem. Soc. 112, 323-333 (1990); Garvey et al., J. Org. Chem. 56, 436 (1990). Further suitable peptidomimetics include hydroxy-1,2,3,4-tetrahydroisoquinoline-3-carboxylate (Miyake et al., J. Takeda Res. Labs 43, 53-76 (1989)); 1,2,3,4-tetrahydroisoquinoline-3-carboxylate (Kazmierski et al., J. Am. Chem. Soc. 133, 2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

The amino acid residues of the peptides can be modified by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26, 294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

Examples of PKs whose activity can be modulated by peptide and peptide derivatives, as described herein, include, but are not limited to, PKs belonging to the following PK families: Src associated kinases, endothelial growth factor receptors, fibroblast growth factor receptors (FGFRs), hepatic growth factor receptors (HGFRs), epidermal growth factor receptors (EGFRs), neural growth factor receptors (NGFRs), Janus kinases (JAKs), Activin receptor-like kinases (ALKs), discoidin domain receptors (DDRs), Ephrin receptors (EphRs), insulin and IGF receptor kinases and Polo family kinases. Suitable members from the Src kinase family include, but are not limited to, Src, Yes, Fyn, Fgr, Lyn, Hck, Lck, Csk and Matk. Suitable members from the endothelial growth factor receptors family include, but are not limited to Tie, Tek, PDGF receptor a and b, Flt 1 and 4 and Flk1. Suitable members from the FGFR family include, but are not limited to, Flg, Bek, FGFR3 and FGFR4. Suitable members from the ALK family include, but are not limited to, ALK1, ALK2, ALK3, ALK4, ALK5, and ALK6. Suitable members from the HGFR family include, but are not limited to, c-Met, c-Sea and Ron. Suitable members from the EGFR family include, but are not limited to, EGFR, ErbB2, ErbB3 and ErbB4. Suitable members from the NGFR family include, but are not limited to, Trk-NGFR, TrkB and TrkC. Suitable members from the JAK family include, but are not limited to, Jak1, Jak2, Jak3 and Tyk2. Suitable members from the DDR family include, but are not limited to, DDR1 and DDR2. Suitable members from the EphR family include, but are not limited to, Eph-B4. Suitable members from the Polo family include, but are not limited to, Plk, Plx1, polo, SNK, CDC5, Sak, Prk, Fnk and Plo1. Other suitable PKs include, but are not limited to, focal adhesion kinase (FAK), c-Ab1, Ret, insulin receptor kinase (IRK), Syk and Zap70, ACK and TEC.

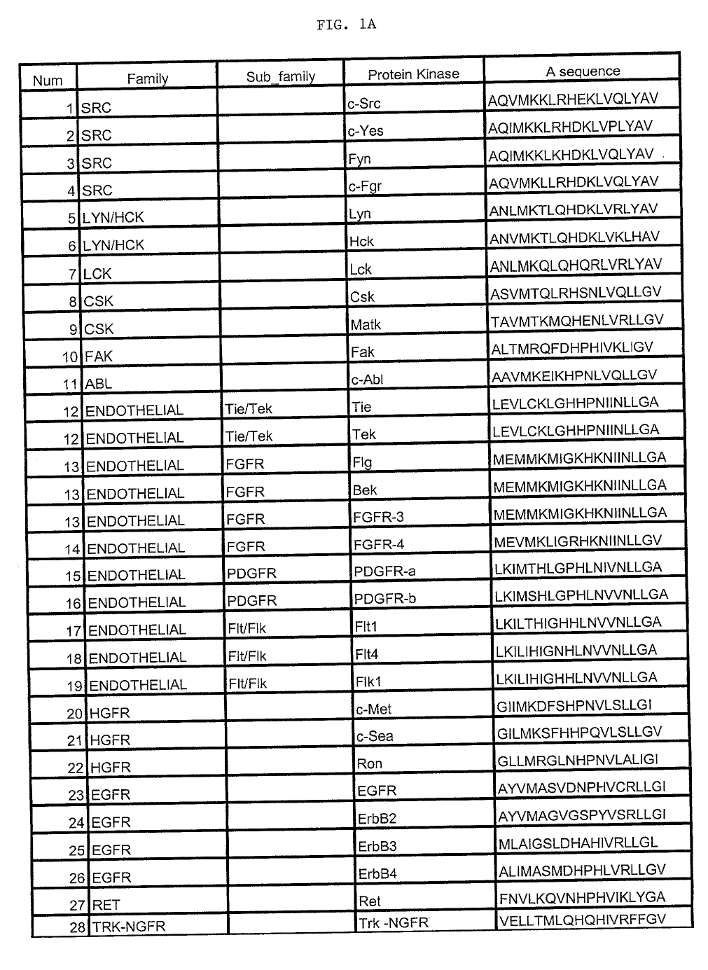

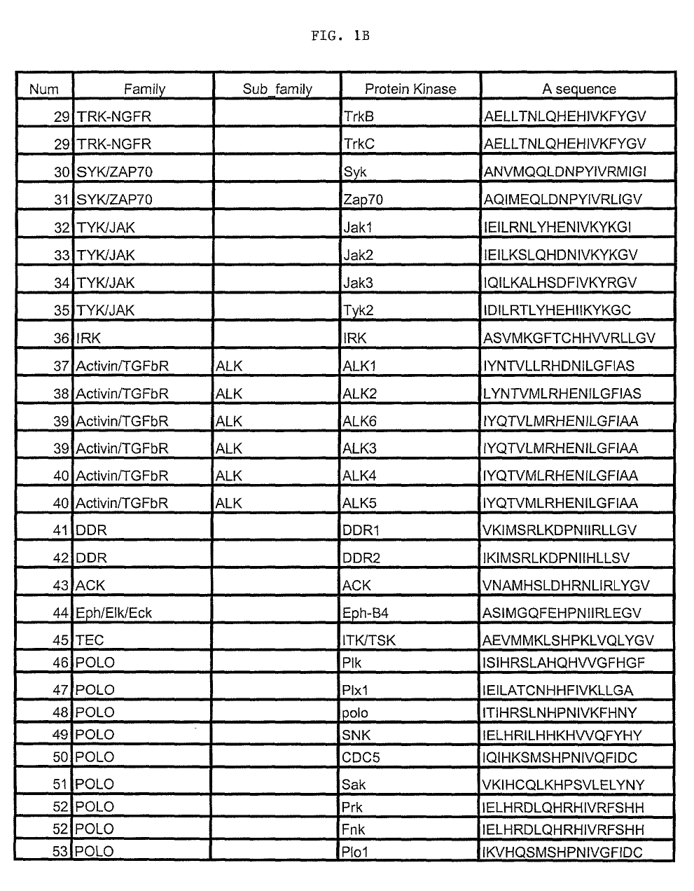

As shown in FIG. 1, the sequences of suitable peptide members of the A region of PKs from different families include, but are not limited to: Src, Yes, Fyn, Fgr, Lyn, Hck, Lck (SEQ ID NO. 1 to 7); Csk and Matk (SEQ ID NO. 8 to 9); focal adhesion kinase (FAK) (SEQ ID NO. 10); c-Abl (SEQ ID NO. 11); endothelial growth factor receptors Tie, Tek, FGF receptor (Flg, Bek, FGFR3, FGFR4), PDGF receptors a and b, Flt 1 and 4 and Flk1 (SEQ ID NO. 12 to 19); HGF receptors c-Met, c-Sea and Ron (SEQ ID NO. 20 to 22); EGF receptor (EGFR, ErbB2, ErbB3, ErbB4) (SEQ ID NO. 23 to 26); Ret (SEQ ID NO. 27); NGF receptors (Trk) (SEQ ID NO. 28 to 29); Syk and Zap70 (SEQ ID NO. 30 to 31); Jak kinases 1 through 3 and Tyk2 (SEQ ID NO. 32 to 35); insulin receptor kinase (IRK) (SEQ ID NO. 36); Activin receptor-like kinases 1 through 6 (ALK1, 2, 3, 4, 5, 6) (SEQ ID NO. 37 to 40); discoidin domain receptors 1 and 2 (DDR) (SEQ ID NO. 41 to 42); ACK (SEQ ID NO. 43); Ephrin receptor B4 (SEQ ID NO. 44); TEC (SEQ ID NO. 45); Polo family kinases Plk, Plx1, polo, SNK, CDC5, Sak, Prk, Fnk and Plo1 (SEQ ID NO.46 to 53).

The amino acid at the N-terminus of the A region is at position 1 and can be referred to as “[AA]₁”. The next amino acid in the sequence, referred to as “[AA]₂”, is at position 2 and is followed by amino acids [AA]₃ through [AA]ₘ, which are at positions 3 to m, where m is the position number of the amino acid at the C-terminus of the A region. Likewise, (m-12) is the position number of the amino acid twelve amino acid residues before the C-terminus of the A region. Thus, a peptide 20-mer with an amino acid sequence [AA₁] through [AA₂₀] includes the first twenty amino acids in the A region. A peptide derivative of the A region with an amino acid sequence [AA₅] through [AA₁₆] includes the fifth amino acid through the sixteenth amino acid in the A region, and a peptide derivative of the A region with an amino acid sequence [AA]₍ₘ₋₁₂₎ through [AA]ₘ includes the last twelve amino acids in the A region. In this invention, m can have a value between 5 and 20.

The present invention includes peptides having amino acid sequences corresponding to the sequence found in the A region of PKs, subsequences thereof and modified subsequences thereof. Examples of suitable subsequences include, but are not limited to, sequences corresponding to [AA₁] through [AAₘ], [AA]₁ through [AA]₁₂, [AA]₅ through [AA]₁₆, [AA]₉ through [AA]₂₀, [AA]₍ₘ₋₁₂₎ through [AA]ₘ, [AA]₍ₘ₋₁₂₎ through [AA]₍ₘ₋₂₎ and [AA]₍ₘ₋₂₀₎ through [AA]₍ₘ₋₈₎ of the A region of a PK, and subsequences thereof. The above designated sequences are preferred.

The present invention includes peptides having amino acid sequences corresponding to a modified sequence or subsequence of the A region of PKs and which modulate the activity of PKs including: Plk; Plx1; polo; SNK; CDC5; Sak; Prk; Plo1; ALK1; ALK2; ALK3; c-Src; c-Yes; Fyn; c-Fgr; Lyn; Hck; Lck; Csk; Matk; Fak; c-Abl; Tie; PDGFR-b; PDGFR-a; Flt1; Flt4; Flg; FGFR-4; c-Met; c-Sea; Ron; EGFR; ErbB2; ErbB3; ErbB4; Ret; Trk-NGFR; TrkB; Syk; Zap70; Jak1; Jak2; Jak3; IRK; DDR1; DDR2; Tyk2; Eph-B4; ITK/TSK and ACK.

In one aspect, one, two or more of the amino acids in the sequence or subsequence are modified with conservative substitutions; the substitutions can be in consensus positions, in non-consensus positions or in both. In another aspect, one, two or more of the amino acids in the sequence or subsequence are modified with severe substitutions; the substitutions are preferably in non-consensus positions. FIG. 2 provides examples of conservative amino acid substitutions for the A region of: Src, Yes, Fyn, Fgr, Lyn, Hck, Lck (SEQ ID NO. 1 to 7); Csk and Matk (SEQ ID NO. 8 to 9); focal adhesion kinase (FAK) (SEQ ID NO. 10); c-Abl (SEQ ID NO. 11); endothelial growth factor receptors Tie, Tek, FGF receptor (Flg, Bek, FGFR3, FGFR4), PDGF receptors a and b, Flt 1 and 4 and Flk1 (SEQ ID NO. 12 to 19); HGF receptors c-Met, c-Sea and Ron (SEQ ID NO. 20 to 22); EGF receptor (EGFR, ErbB2, ErbB3, ErbB4) (SEQ ID NO. 23 to 26); Ret (SEQ ID NO. 27); NGF receptors (Trk) (SEQ ID NO. 28 to 29); Syk and Zap70 (SEQ ID NO. 30 to 31); Jak kinases 1 through 3 and Tyk2 (SEQ ID NO. 32 to 35); insulin receptor kinase (IRK) (SEQ ID NO. 36); Activin receptor-like kinases 1 through 6 (ALK1, 2, 3, 4, 5, 6) (SEQ ID NO. 37 to 40); discoidin domain receptors 1 and 2 (DDR) (SEQ ID NO. 41 to 42); ACK (SEQ ID NO. 43); Ephrin receptor B4 (SEQ ID NO. 44); TEC (SEQ ID NO. 45); Polo family kinases Plk, Plx1, polo, SNK, CDC5, Sak, Prk, Fnk and Plo1 (SEQ ID NO. 46 to 53).

The conservative substitutions can occur by exchanging amino acids with aligned A region sequences, as shown in FIG. 2, as well as by substituting the listed amino acids that are not associated with a known A region sequence.

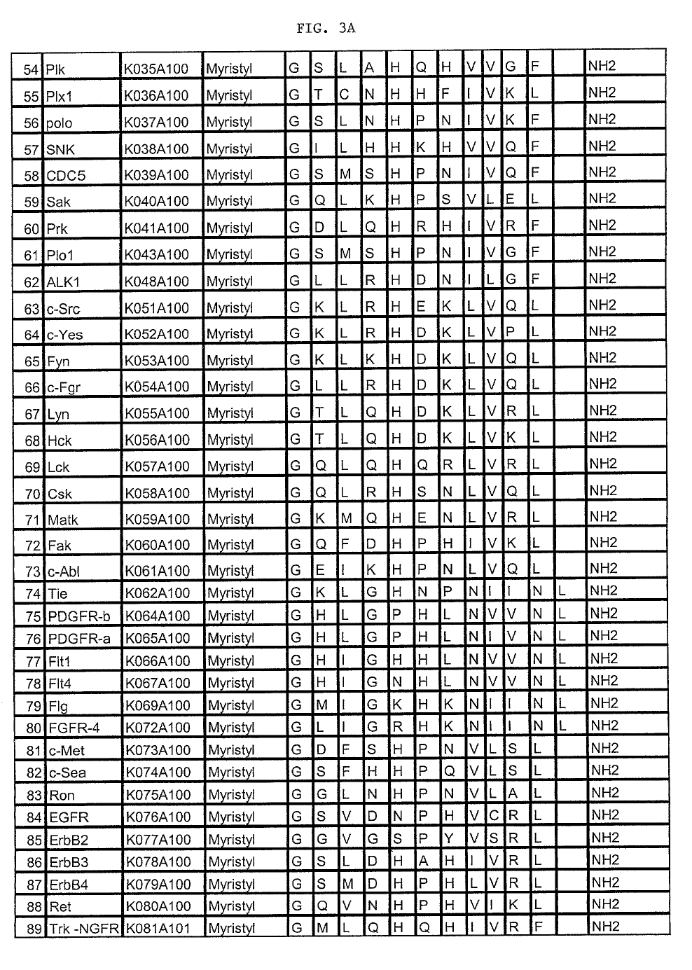

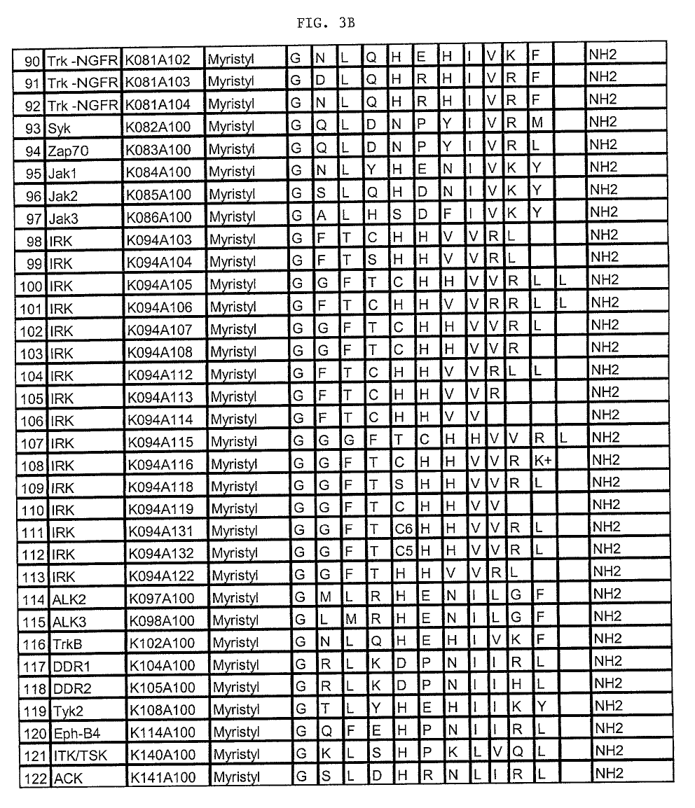

Specific examples of peptide derivatives of the present invention include peptides: Plk K035A100; Plx1 K036A100; polo K037A100; SNK K038A100; CDC5 K039A100; Sak K040A100; Prk K041A100; Plo1 K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Abl K061A100; Tie K062A100; PDGFR-b K064A100; PDGFR-a K065A100; Flt1 K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K081A104; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A117 K094A118 K094A 119 K094A131 K094A132 K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively), as specified in FIG. 3.

The N-terminus and/or C-terminus of these peptides can be modified, as described above and as shown in FIG. 3. The N-terminal of these peptides is myristylated and the C-terminal is amidated. Other protecting groups for amides and carboxylic acids can be used, as described above. Optionally, one or both protecting groups can be omitted. The peptides may be linear or cyclic.

Also included are peptides having the sequence of: Plk K035A100; Plx1 K036A100; polo K037A100; SNK K038A100; CDC5 K039A100; Sak K040A100; Prk K041A100; Plo1 K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Abl K061A100; Tie K062A100; PDGFR-b K064A100; PDGFR-a K065A100; Flt1 K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K081A104; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A117 K094A118 K094A 119 K094A131 K094A132 K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively), as specified in FIG. 3, with the proviso that any one or two of the amino residues in the peptide can vary, being replaced by any naturally occurring amino acid or analog thereof.

The present invention also includes cyclic peptides having amino acid sequences corresponding to a modified sequence or subsequence of the A region of PKs. These cyclic peptides modulate the activity of PKs.

A “cyclic peptide” refers, in one instance, to a peptide or peptide derivative in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the peptide, preferably the side chain of the C-terminal amino acid. For example, an amide can be formed between the nitrogen atom at the N-terminus and the carbonyl carbon in the side chain of an aspartic acid or a glutamic acid. Alternatively, the peptide or peptide derivative can be cyclized by forming a covalent bond between the carbonyl at the C-terminus of the compound and the side chain of a suitable amino acid in the peptide, preferably the side chain of the N-terminal amino acid. For example, an amide can be formed between the carbonyl carbon at the C-terminus and the amino nitrogen atom in the side chain of a lysine or an ornithine. Additionally, the peptide or peptide derivative can be cyclized by forming an ester between the carbonyl carbon at the C-terminus and the hydroxyl oxygen atom in the side chain of a serine or a threonine.

“Cyclized” also refers to forming a ring by a covalent bond between the side chains of two suitable amino acids in the peptide, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an ornithine.

In addition, a peptide or peptide derivative can be cyclized with a linking group between the two termini, between one terminus and the side chain of an amino acid in the peptide or peptide derivative, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

Suitable substitutions or insertions in the original A region amino acid sequence or subsequence are those which result in a peptide derivative, as defined above, which modulates the activity of a PK. The activity of a PK is “modulated” when the activity of the PK is increased or decreased. An increase or decrease in the activity of a PK can be detected by assessing in vitro the extent of phosphorylation of a protein substrate of the PK being tested or by a corresponding modulation, i.e., increase or decrease, in a cellular activity or function which is under the control of the PK. Examples of these cellular functions include cell proliferation, cell differentiation, cell morphology, cell survival or apoptosis, cell response to external stimuli, gene expression, lipid metabolism, glycogen or glucose metabolism and mitosis.

It can be readily determined whether a peptide or peptide derivative modulates the activity of a PK by incubating the peptide or peptide derivative with cells which have one or more cellular activities controlled by the PK. The cells are incubated with the peptide or peptide derivative to produce a test mixture under conditions suitable for assessing the activity of the protein kinase. The activity of the PK is assessed and compared with a suitable control, e.g., the activity of the same cells incubated under the same conditions in the absence of the peptide or peptide derivative. A greater or lesser activity of the PK in the test mixture compared with the control indicates that the test peptide or peptide derivative modulates the activity of the PK.

Suitable cells for the assay include normal cells which express a membrane bound or intracellular PK, cells which have been genetically engineered to express a PK, malignant cells expressing a PK or immortalized cells which express a PK.

Conditions suitable for assessing PK activity include conditions suitable for assessing a cellular activity or function under control of the PK. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors).

In another aspect, the activity of certain PK (e.g., Akt/PKB, Dudek et al., Science 275:661 (1997)) can be evaluated by growing the cells under serum deprivation conditions. Cells are typically grown in culture in the presence of a serum such as bovine serum, horse serum or fetal calf serum. Many cells, for example, nerve cells such as PC-12 cells, generally do not survive with insufficient serum. The use of insufficient serum to culture cells is referred to as “serum deprivation conditions” and includes, for example, from 0% to about 4% serum. PK activity is determined by the extent to which a peptide pepteptide derivative can protect cells, e.g., neuronal cells, from the consequences of serum deprivation. Specific conditions are provided in Dudek et al., and in Example 4 of the application entitled “SHORT PEPTIDES WHICH SELECTIVELY MODULATE INTRACELLULAR SIGNALING” (filed on May 21, 1997, U.S. application Ser. No. 08/861,153), the pertinent teachings of which are incorporated herein by reference.

Generally, the activity of the PK in the test mixture is assessed by making a quantitative measure of the cellular activity which the PK controls. The cellular activity can be, for example, cell proliferation. Examples of cells in which proliferation is controlled by a PK include endothelial cells such as bovine aortic cells, mouse MSI cells or mouse SVR cells (see Arbiser et al., Proc. Natl. Acad. Sci. USA 94:861 (1997)), vascular smooth muscle cells, fibroblasts of various tissue origin, and malignant cells of various tissues such as breast cancer, lung cancer, colon cancer, prostate cancer or melanoma. PK activity is assessed by measuring cellular proliferation, for example, by comparing the number of cells present after a given period of time with the number of cells originally present. One example of PKs having to do with cellular proliferation are the receptors of the activin-like kinases (ALKs) super-family.

If cells are being used in which the PK controls cell differentiation (e.g., PC-12 cells transfected with c-Src, see Alema et al., Nature 316:557 (1985)), activity is assessed by measuring the degree of differentiation. Activity can be assessed the degree to which neurites are extended and the degree to which markers of neuronal differentiation are expressed in PC-12 cells transfected with c-Src; see Alema et al., and the degree to which the formation of mesoderm in developing Xenopus embroya cells is induced; see Burgess and Maciag, Ann. Rev. Biochem., 58:575 (1989) and Dionne et al., WO 92/00999. Activity can also be assessed by the extent to which gene expression, cell morphology or cellular phenotype is altered (e.g., the degree to which cell shape is altered or the degree to which the cells assume a spindle-like structure). One example of a change in cellular morphology is reported in the application entitled “SHORT PEPTIDES WHICH SELECTIVELY MODULATE INTRACELLULAR SIGNALING” (filed on May 21, 1997, U.S. application Ser. No. 08/861,153), which discloses that certain peptide derivatives of the HJ loop of protein tyrosine kinases can cause vascular smooth muscle cells to become elongated and assume a spindle-like shape.

It is to be understood that the assay described hereinabove for determining whether a peptide or peptide derivative modulates a cellular activity or function under the control of a PK can be performed with cells other than those specifically described herein. PKs not yet discovered or PKs whose function is not yet known can also be used in this assay, once it has been determined which cellular functions or activities they control. These PKs are also within the scope of the present invention.

The present invention is also directed to a method of modulating the activity of a protein kinase in a subject. A “subject” is preferably a human, but can also be animals in need of treatment, e.g., veterinary animals (e.g., dogs, cats, and the like), farm animals (e.g., cows, pigs, horses, chickens and the like) and laboratory animals (e.g., rats, mice, guinea pigs and the like).

The activity of a PK in a subject can be modulated for the purpose of treating diseases that are caused by over activity or under activity of PKs. For example, inhibition of c-Met or tyrosine kinase receptors which respond to fibroblast growth factor (FGF) or vascular endothelial growth factor (VEGF) decreases angiogenesis. In addition, RET is involved in certain thyroid cancers. Peptides and peptide derivatives of the present invention which modulate the activity of these enzymes can be used to treat cancer in a subject when administered to the subject in a therapeutically effective amount.

Restenosis is caused by vascular smooth muscle proliferation in response to, for example, vascular injury caused by balloon catheterization. Vascular smooth muscle proliferation is also a cause of atherosclerosis. Vascular smooth muscle proliferation is a result of, for example, inhibition of CSK and/or stimulation of tyrosine kinase receptors which respond to FGF or platelet derived growth factor (PDGF). Thus, restenosis and atherosclerosis can be treated with a therapeutically effective amount of a peptide or peptide derivative which inhibits tyrosine kinase receptors which respond to FGF or PDGF or which activate CSK.

FGF has also been implicated in psoriasis, arthritis and benign prostatic hypertrophy (Dionne et al., WO 92/00999). These conditions can be treated with A peptides from PKs which respond to FGF.

Src activity is responsible, at least in part, for bone resorption. Thus, osteoporosis can be treated with a therapeutically effective amount of a peptide or peptide derivative which inhibits Src activity or which activates Csk.

Lyn and HCK are activated during the non-specific immune response which occurs in individuals with arthritis, as a result of autoimmune responses. Lyn is also activated in individuals with septic shock. Thus, these conditions can be treated with a therapeutically effective amount of a peptide or peptide derivative which inhibits the activity of these PKs.

Lck is expressed in T cells and is activated during a T cell immune response. Similarly, Lyn is expressed in B cells and activated during a B cell immune response. Thus, conditions which are caused by overactivation of T cells or B cells can be treated by administering a therapeutically effective amount of a peptide or peptide derivative which inhibits Lck or Lyn, respectively. Conditions which are caused by underactivation of T cells or B cells can be treated by administering a therapeutically effective amount of a peptide or peptide derivative which stimulates Lck or Lyn, respectively. In addition, a severe reduction of the B cell progenitor kinase leads to human X-linked agammaglobulinemia, which can be treated by administering a therapeutically effective amount of a peptide or peptide derivative which stimulates B cell progenitor kinase. Decreased function of other PKs can also lead to disease. For example, a decrease in the activity of insulin receptor tyrosine kinase (IRK) is a cause of various types of diabetes. These types of diabetes can be treated by administering a therapeutically effective amount of a peptide or peptide derivative which increases the activity of the IRK.

Another family of transmembrane protein kinases is composed of members of the TGFβ/Activin/BMP receptors which transduce signals of the corresponding cytokines. The TGFβ/Activin/BMP cytokines participate in various processes of tissue remodelling, including the induction of bone formation, hair growth, adipose tissue proliferation, neural cell stimulation and differentiation of pancreatic islet cells. Therefore, modulation of the activity of these receptor kinases can assist tissue repair, inhibit tissue fibrosis and fat tissue growth, assist in hair growth, induce differentiation of pancreatic β-cells, help neural cell survival and function and enhance bone formation.

Based on methods disclosed herein, peptides and peptide derivatives can be designed to modulate the activity of PKs whose A region has been sequenced or will be sequenced in the future and whose cellular function is known. As a consequence, peptides and peptide derivatives can be designed to affect (increase or decrease) those cellular functions. It is possible that future research will reveal that certain disease conditions, whose underlying causes are presently unknown, are brought about by the overactivity or underactivity of cellular functions controlled by these PKs. These diseases can be treated by administering peptides which are peptide derivatives of the A region of the overactive or underactive PK. Suitable peptides and peptide derivatives can be identified by methods disclosed herein. These methods of treatment, peptides and peptide derivatives are encompassed within the scope of the present invention. A “therapeutically effective amount” is the quantity of compound which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment. With respect to cancer, an “improved clinical outcome” includes a longer life expectancy. It can also include slowing or arresting the rate of growth of a tumor, causing a shrinkage in the size of the tumor, a decreased rate of metastasis and/or improved quality of life (e.g., a decrease in physical discomfort or an increase in mobility).

With respect to diabetes, an improved clinical outcome refers to a longer life expectancy, a reduction in the complications of the disease (e.g., neuropathy, retinopathy, nephropathy and degeneration of blood vessels) and an improved quality of life, as described above. Another aspect of an improved clinical outcome is a reduction in medication dosage (e.g., a reduction in insulin or other hypoglycemic agent needed to maintain adequate blood glucose levels).

With respect to obesity, an improved clinical outcome refers to increased weight reduction per caloric intake or a reduction in food intake. It also refers to a decrease in the complications which are a consequence of obesity, for example heart disease such as arteriosclerosis and high blood pressure.

The amount of peptide or peptide derivative administered to the individual will depend on the type and severity of the disease and on the characteristics of the individual, such as general health, age, sex, body weight and tolerance to drugs. The skilled artisan will be able to determine appropriate dosages depending on these and other factors. Typically, a therapeutically effective amount of the peptide or peptide derivative can range from about 1 mg per day to about 1000 mg per day for an adult. Preferably, the dosage ranges from about 1 mg per day to about 100 mg per day.

The peptide and peptide derivatives of the present invention are preferably administered parenterally. Parenteral administration can include, for example, systemic administration, such as by intramuscular, intravenous, subcutaneous, or intraperitoneal injection. Peptides or peptide derivatives which resist proteolysis can be administered orally, for example, in capsules, suspensions or tablets. The peptide or peptide derivative can also be administered by inhalation or insufflation or via a nasal spray.

The peptide or peptide derivative can be administered to the individual in conjunction with an acceptable pharmaceutical carrier as part of a pharmaceutical composition for treating the diseases discussed above. Suitable pharmaceutical carriers may contain inert ingredients which do not interact with the peptide or peptide derivative. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release of Biological Active Agents, John Wiley and Sons, 1986).

The peptide and peptide derivatives of the present invention have many utilities other than as a therapeutic agent. Some of these uses are discussed in the following paragraphs.

The A region peptides of the present invention are derived from an array which is linear in the native protein. These peptides can be useful in the preparation of specific antibodies against PKs. Moreover, since the A region sequence is unique to each sub-family of PK, anti-A region antibodies can be specifically used to isolate distinct sub-families of PK.

Suitable antibodies can be raised against an A region peptide by conjugating the peptide to a suitable carrier, such as keyhole limpet hemocyanin or serum albumin; polyclonal and monoclonal antibody production can be performed using any suitable technique. A variety of methods have been described (see e.g., Kohler et al., Nature, 256: 495-497 (1975) and Eur. J. Immunol. 6:511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Antibodies, including monoclonal antibodies, against A region peptides have a variety of uses. For example, those against or reactive with the protein from which the A peptides was derived, and preferably which bind specifically to said protein, can be used to identify and/or sort cells exhibiting that protein on the cell surface (e.g., by means of fluorescence activated cell sorting or histological analyses). Monoclonal antibodies specific for the protein can also be used to detect and/or quantitate the protein expressed on the surface of a cell or present in a sample (e.g., in an ELISA). Alternatively, the antibodies can be used to determine if an intracellular PK is present in the cytoplasm of the cell. A lysate of the cell is generated (for example, by treating the cells with sodium hydroxide (0.2 N) and sodium dodecyl sulfate (1%) or with a non-ionic detergent like NP-40, centrifugating and separating the supernatant from the pellet), and treated with anti-A region antibody specific for the PK. The lysate is then analyzed, for example, by Western blotting or immunoprecipitation for complexes between PK and antibody. Some PKs become membrane-bound or cytoskeleton-associated following stimulation. Anti-A region antibodies can be utilized for the study of the intracellular distribution (compartmentalization) of various subfamilies of PKs under various physiological conditions via the application of conventional immunocytochemistry such as immunofluorescence, immunoperoxidase technique and immunoelectron microscopy, in conjunction with the specific anti-A region antibody.

Antibodies reactive with the A region are also useful to detect and/or quantitate the PK or A peptide in a sample, or to purify the PK from which the A region was derived (e.g., by immunoaffinity purification).

The A region within PKs plays a key role in regulating the activity of PKs, as is evidenced by the fact that the peptides and peptide derivatives of the present invention have such a dramatic effect on the activity of PKs. The A region peptides of the present invention can also be used to identify ligands which interact with the A regions of specific PKs and which modulate the activity PKs. For example, an affinity column can be prepared to which a specific A region peptide is covalently attached, directly or via a linker. This column, in turn, can be utilized for the isolation and identification of specific ligands which bind the A region peptide and which will also likely bind the PK from which the A region peptide was derived. The ligand can then be eluted from the column, characterized and tested for its ability to modulate PK function.

Peptide sequences in the compounds of the present invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these references are incorporated herein by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd° and Acm is easily removed by iodine treatment.

The invention is illustrated by the following examples which are not intended to be limiting in any way.

### EXAMPLE 1

Preparation of “A Peptides”

The novel compounds of this invention can be synthesized utilizing a 430A Peptide Synthesizer from Applied Biosystems using F-Moc technology according to manufacturer's protocols. Other suitable methodologies for preparing peptides are known to person skilled in the art. See e.g., Merrifield, R. B., Science, 232: 341 (1986); Carpino, L. A., Han, G. Y., J. Org. Chem., 37: 3404 (1972); Gauspohl, H., et al., Synthesis, 5: 315 (1992)), the teachings of which are incorporated herein by reference. Rink Amide Resin [4(2′, 4′ Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzotriazolyl-1-yl)-1,1,3,3-tetramethyluronium) dissolved in HOBt (1-hydroxybenzotriazole) and DMF (dimethylformamide). The amino acid was dissolved in this solution with additional NMP. DIEA (diisopropylethylamine) was added to initiate activation. Alternatively, the activation method of DCC (dicyclohexylcarbodiimide) and HOBt was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2-ethandithiol); 0.5 ml thioanisole; 0.5 ml D.I. H₂O; 10 ml TFA.

### EXAMPLE 2

Glucose Uptake by Adipose Tissue Cells

1. Materials

30 ml plastic bottle (Nalgene 2103-0001)

50 ml plastic conical tube (Miniplast 204-21)

TC tubes (Nunc 146183)

Test tubes (Sarstedt 72.7000)

250μ nylon mesh

Collagenase Type 1 (Worthington CLS 4196)

Dinonyl phthalate (Merck 1.09669.0100)

3 H-Deoxy Glucose (ICN 27088S.2), 30 Ci/mmole, 0.25 mCi, 0.25 ml

2. Solutions

Krebs Ringer Bicarbonate HEPES buffer, containing 1% bovine fraction 5 albumin and 200 nM adenosine was made, using stock solutions:

Stock solution 1 - salts

| | |
|---|---|
| 120 mM | 35.04 NaCl |
| 4 mM | 2.73 g KH₂PO₄ |
| 1 mM | 0.55 g CaCl₂ (0.74 g CaCl₂ · 2H₂O) Dissolved in a small |
| | flask and added to other salts. |

Stock solution 2 - Sodium bicarbonate

| | |
|---|---|
| 10 mM | 4.2 g NaHCO₃; dissolved in a 500 ml volumetric flask. |

Stock solution 3 - HEPES

| | |
|---|---|
| 30 mM | 35.75 g HEPES (39.05 g HEPES Sodium salt); dissolved in a |
| | 500 ml volumetric flask pH to 7.4 before being brought up to |
| | volume. |

10 ml of each stock solution (1,2 and 3) was used per 100 ml double distilled water on day of use.

Stock solution 4—Adenosine (2 mM)

3. Adipose Cell Isolation Procedure

To 3 ml of buffer with 10 mg collagenase, 3 g epididymal fat pad (from 2-3 male rats) was introduced. The fat was cut up with scissors. The pieces of fat were swirled and shaken in the collagenase solution in a 37° C. water bath, set at 100-150 repetitions/minute, for approximately 1 hour with swirling every 15 minutes while digesting and every 5 minutes towards the end. About 6 ml of buffer was then added to the vial. A 250μ nylon mesh was placed over the top of the vial and secured with a rubber band. The contents of the vial were gently squeezed through the mesh into a 50 ml plastic tube. The residual fat tissue was washed twice and the contents of each wash were filtered through the mesh into the 50 ml plastic tube. The total volume for each wash was 15 ml. The tube was centrifuged whereby the adipose cells floated at the top of the liquid. The buffer was removed using a 35 ml metal-tipped syringe with a needle. Buffer was added to 15 ml and clumps of adipose cells were gently broken up by mixing up and down in the syringe. This procedure was repeated for a total of 4 centrifugations at 1000 rpm with the last centrifugation at 2000 rpm. At this point, any free fat was removed from the top of the cells. Buffer was added to the cell suspension and the cell suspension with buffer was diluted and removed to form a cytocrit of 5-10%. The cells were kept at 37° C. for 1 hour.

4. Glucose Uptake Procedure

500 μl buffer was placed with or without additives (insulin 10-10,000 μU/ml, peptides 0.1-10 μM) in 10 ml plastic tubes. 500 μl aliquots of the cell suspension were added to the tubes. After incubation for 30 minutes at 37° C. in a shaking water bath (approximately 300 strokes/minute), 200 μl of buffer containing 3H-Deoxy Glucose (approx. 1200 cpm μl) was added to each tube. After 30 minutes incubation with the 3H-DOG at 37° C., 200 μl aliquots were transferred to microcentrifuge tubes containing 200 μl Dinonyl phthalate. Cells were rapidly separated from the aqueous buffer by centrifugation at 10,000 g for 30-60 sec. The cells were separated in the top layer from the aqueous buffer by the presence of Dinonyl phthalate.

Cell associated radioactivity was counted in a liquid scintillation counter.

Increase of Glucose-Uptake by IRK-Derived Peptide

Glucose-uptake was measured in fresh adipocytes, incubated with or without insulin (10 μU) as described above, in the absence (control) or the presence of 10 μM of peptide K094A107 (derived from the A region of IRK), which was kept in a reducing environment (5 to 25 μM DTT).

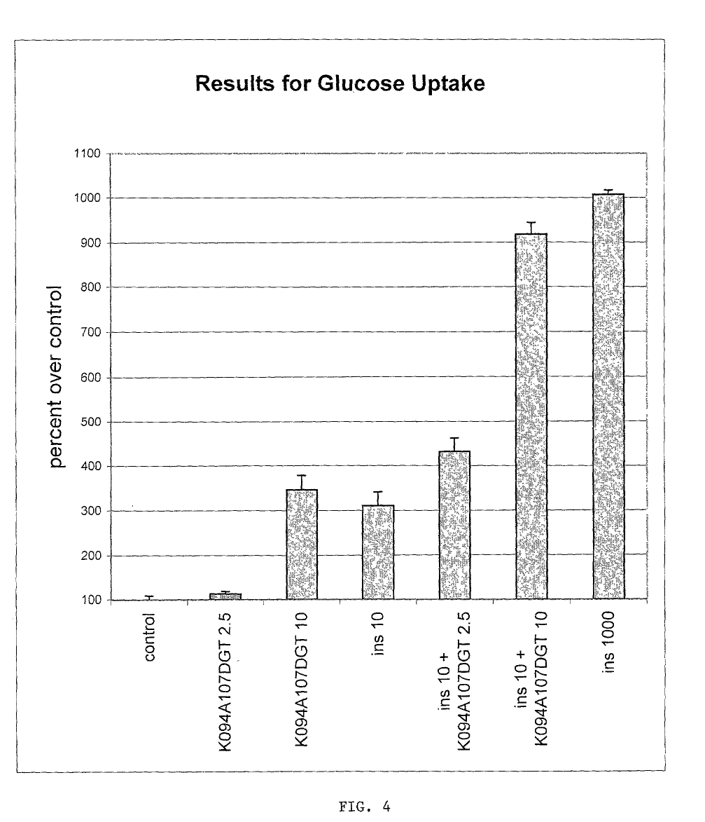

The results of this uptake are shown in FIG. 4.

These results show that peptides from the A region of IRK increase the uptake of glucose by adipocytes in the absence of insulin, and act synergistically with insulin to further enhance insulin-directed glucose uptake.

While this invention has been particularly shown and described with references to preferred embodiments thereof, it will be understood by those skilled in the art that various changes in form and details may be made therein without departing from the scope of the invention encompassed by the appended claims.

What is claimed is:

1. A peptide consisting of a peptide derivative of the A region of a protein kinase, wherein:
a) said peptide has between about five and about twenty amino acids or conservatively substituted amino acids; and
b) said peptide modulates activity of the protein kinase.

2. The peptide of claim 1 wherein the peptide is cyclic.

3. The peptide of claim 1 wherein the peptide is linear.

4. The peptide of claim 3 wherein the N-terminus and the C-terminus of the peptide are unsubstituted.

5. The peptide of claim 3 wherein at least one of the N-terminus or the C-terminus is substituted.

6. The peptide of claim 5 wherein the N-terminus is amidated and the C-terminus is acylated.

7. The peptide of claim 3 wherein the peptide derivative has the amino acid sequence of any subsequence of the amino acid sequence of said A region of said protein kinase, with the proviso that any one amino acid in the sequence of the peptide derivative can vary, being any amino acid or conservatively substituted amino acid thereof.

8. The peptide of claim 3 wherein the protein kinase is member of a protein kinase family selected from the group of families consisting of Src family kinases, Csk family kinases, endothelial growth factor receptor kinases, fibroblast growth factor receptor kinases, Tyk/Jak kinases, insulin receptor kinases, activin receptor-like kinases, neurotrophin growth factor receptor kinases, discoidin domain receptor kinases, c-Abl, hepatic growth factor receptor kinases, epidermal growth factor receptor kinases, Ret kinase, Syk/Zap kinases and ephrin receptor kinases.

9. The peptide of claim 8 wherein the protein kinase is a Src family kinase selected from the group consisting of c-Src, c-Yes, Fyn, C-Fgr, Lyn, Hck and Lck.

10. The peptide of claim 8 wherein the protein kinase is a Csk family kinase selected from the group consisting of Csk and Matk.

11. The peptide of claim 8 wherein the protein kinase is an endothelial growth factor receptor kinase selected from the group consisting of Tie, Tek, PDGFR-b, PDGFR-a, Flt1, Flt4 and Flk1.

12. The peptide of claim 8 wherein the protein kinase is a fibroblast growth factor receptor kinase selected from the group consisting of Flg, Bek, FGFR-3 and FGFR-4.

13. The peptide of claim 8 wherein the protein kinase is a Tyk/Jak kinase selected from the group consisting of Jak1, Jak2, Jak3 and Tyk2.

14. The peptide of claim 8 wherein the protein kinase is a discoidin domain receptor kinase selected from the group consisting of DDR1 and DDR2.

15. The peptide of claim 8 wherein the protein kinase is an activin receptor-like kinase selected from the group consisting of ALK1, ALK2, ALK3, ALK4, ALK5 and ALK6.

16. The peptide of claim 8 wherein the protein kinase is a neurotrophin receptor kinase selected from the group consisting of Trk-NGFR, TrkB, and TrkC.

17. The peptide of claim 8 wherein the protein kinase is IRK.

18. The peptide of claim 8 wherein the protein kinase is Ret.

19. The peptide of claim 8 wherein the protein kinase is c-Abl.

20. The peptide of claim 8 wherein the protein kinase is a hepatic growth factor receptor kinase selected from the group consisting of c-Met, c-Sea, and Ron.

21. The peptide of claim 8 wherein the protein kinase is an epidermal growth factor receptor kinase selected from the group consisting of EGFR, ErbB2, ErbB3 and ErbB4.

22. The peptide of claim 8 wherein the protein kinase is a Syk/Zap kinase selected from the group consisting of Syk and Zap70.

23. The peptide of claim 8 wherein the protein kinase is Eph-B4.

24. The peptide of claim 3 wherein the peptide derivative has the amino acid sequence of any subsequence of the amino acid sequence of said A region.

25. The peptide of claim 3 wherein the peptide has the sequence of Plk K035A100; Plx1K036A100; polo K037A100; SNK K038A100; CDC5 K039A100; Sak K040A100; Prk K041A100; Plo1K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Ab1K061A100; Tie K062A100; PDGFR-b K064A100; PDGFR-a K065A100; Fltl K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K081A104; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A117 K094A118 K094A119 K094A131 K094A132 K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively), as specified in FIG. 3.

26. A peptide having the sequence of Plk K035A100; Plx1 K036A100; polo K037A100; SNK K038A100; CDC5 K039A100; Sak K040A100; Prk K041A100; Plo1 K043A100; ALK1 K048A100; c-Src K051A100; c-Yes K052A100; Fyn K053A100; c-Fgr K054A100; Lyn K055A100; Hck K056A100; Lck K057A100; Csk K058A100; Matk K059A100; Fak K060A100; c-Abl K061A100; Tie K062A100; PDGFR-b K064A100; PDGFR-a K065A100; Flt1 K066A100; Flt4 K067A100; Flg K069A100; FGFR-4 K072A100; c-Met K073A100; c-Sea K074A100; Ron K075A100; EGFR K076A100; ErbB2 K077A100; ErbB3 K078A100; ErbB4 K079A100; Ret K080A100; Trk-NGFR K081A101 K081A102 K081A103 K081A104; Syk K082A100; Zap70 K083A100; Jak1 K084A100; Jak2 K085A100; Jak3 K086A100; IRK K094A103 K094A104 K094A105 K094A106 K094A107 K094A108 K094A112 K094A113 K094A114 K094A115 K094A116 K094A117 K094A118 K094A119 K094A131 K094A132 K094A122; ALK2 K097A100; ALK3 K098A100; TrkB K102A100; DDR1 K104A100; DDR2 K105A100; Tyk2 K108A100; Eph-B4 K114A100; ITK/TSK K140A100; ACK K141A100 (SEQ ID NO. 54 to 122, respectively), as specified in FIG. 3, with the proviso that any one amino acid residue in the peptide can vary, being any naturally occurring amino acid or conservatively substituted amino acid thereof.

27. A peptide comprising a sequence of amino acids AA₁ through AA₁₈ or a subsequence thereof comprising at least five amino acids, wherein:
AA₁ is selected from the group consisting of alanine and glycine;
AA₂ is selected from the group consisting of glutamine, asparagine, glutamic acid, aspartic acid and an aliphatic, substituted aliphatic, benzyl, substituted benzyl, aromatic or substituted aromatic ester of a glutamic or aspartic acid;
AA₃ is selected from the group consisting of valine, isoleucine, leucine and methionine;
AA₄ is selected from the group consisting of methionine, isoleucine, leucine and valine;
AA₅ is selected from the group consisting of lysine and arginine;
AA₆ is selected from the group consisting of lysine, leucine, threonine, glutamine, arginine, isoleucine, methionine, valine, serine, glutamic acid, asparagine, aspartic acid and an aliphatic, substituted aliphatic, benzyl, substituted benzyl, aromatic or substituted aromatic ester of a glutamic or aspartic acid;
AA₇ is selected from the group consisting of leucine, isoleucine, methionine, and valine;
AA₈ is selected from the group consisting of arginine, lysine, glutamine, glutamic acid, aspartic acid, asparagine and an aliphatic, substituted aliphatic, benzyl, substituted benzyl, aromatic or substituted aromatic ester of a glutamic or aspartic acid;
AA₉ is histidine;
AA₁₀ is selected from the group consisting of glutamic acid, aspartic acid, glutamine, asparagine and an aliphatic, substituted aliphatic, benzyl, substituted benzyl, aromatic or substituted aromatic ester of a glutamic or aspartic acid;
AA₁₁ is selected from the group consisting of lysine and arginine;
AA₁₂ is selected from the group consisting of leucine, isoleucine, methionine and valine;
AA₁₃ is selected from the group consisting of valine, isoleucine, leucine and methionine;
AA₁₄ is selected from the group consisting of glutamine, proline, arginine, lysine, glutamic acid, aspartic acid, asparagine and an aliphatic, substituted aliphatic, benzyl, substituted benzyl, aromatic or substituted aromatic ester of a glutamic or aspartic acid;
AA₁₅ is selected from the group consisting of leucine, isoleucine, methionine and valine;
AA₁₆ is selected from the group consisting of tyrosine, histidine, phenylalanine and tryptophan;
AA₁₇ is selected from the group consisting of alanine and glycine; and
AA₈ is selected from the group consisting of valine, isoleucine, leucine and methionine.

28. The peptide of claim 27 wherein the sequence AA₁ through AA₁₈ or a subsequence thereof corresponds to a sequence of the A region of a SRC, LYN, HCK or LCK protein kinase selected from the group consisting of SEQ ID NO: 1, SEQ ID NO:2, SEQ ID NO:3, SEQ ID NO:4, SEQ ID NO:5, SEQ ID NO:6, and SEQ ID NO:7 or a subsequence thereof, with the proviso that any two amino acids in the sequence AA₁ through AA₁₈ or the subsequence thereof can vary according to the listing in claim 27.

29. The peptide of claim 27 wherein the sequence AA₁ through AA₁₈ or a subsequence thereof corresponds to a sequence of the A region of a SRC, LYN, HCK or LCK protein kinase selected from the group consisting of SEQ ID NO: 1, SEQ ID NO:2, SEQ ID NO:3, SEQ ID NO:4, SEQ ID NO:5, SEQ ID NO:6, and SEQ ID NO:7 or a subsequence thereof, with the proviso that any one amino acid in the sequence AA₁ through AA₁₈ or the subsequence thereof can vary according to the listing in claim 27.

30. A peptide comprising a sequence of amino acids AA₁ through AA₁₈ or a subsequence thereof comprising at least five amino acids, wherein:
AA₁ is selected from the group consisting of alanine, threonine, glycine and serine;
AA₂ is selected from the group consisting of serine, alanine, threonine and glycine;
AA₃ is selected from the group consisting of valine, isoleucine, leucine and methionine;
AA₄ is selected from the group consisting of methionine, isoleucine, leucine and valine;
AA₅ is selected from the group consisting of threonine and serine;
AA₆ is selected from the group consisting of glutamine, lysine, glutamic acid, aspartic acid, asparagine, arginine and an aliphatic, substituted aliphatic, benzyl, substituted benzyl, aromatic or substituted aromatic ester of a glutamic or aspartic acid;
AA₇ is selected from the group consisting of leucine, methionine, isoleucine and valine;
AA₈ is selected from the group consisting of arginine, glutamine, lysine, glutamic acid, aspartic acid, asparagine and an aliphatic, substituted aliphatic, benzyl, substituted benzyl, aromatic or substituted aromatic ester of a glutamic or aspartic acid;
AA₉ is histidine;
AA₁₀ is selected from the group consisting of serine, glutamic acid, threonine, aspartic acid, glutamine, asparagine and an aliphatic, substituted aliphatic, benzyl, substituted benzyl, aromatic or substituted aromatic ester of a glutamic or aspartic acid;
AA₁₁ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₂ is selected from the group consisting of leucine, isoleucine, methionine and valine;
AA₁₃ is selected from the group consisting of valine, isoleucine, leucine and methionine;
AA₁₄ is selected from the group consisting of glutamine, arginine, glutamic acid, aspartic acid, asparagine, lysine and an aliphatic, substituted aliphatic, benzyl, substituted benzyl, aromatic or substituted aromatic ester of a glutamic or aspartic acid;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine and alanine; and
AA₁₈ is selected from the group consisting of valine, isoleucine, leucine and methionine.

31. The peptide of claim 30 wherein the sequence AA₁ through AA₁₈ or a subsequence thereof corresponds to a sequence of the A region of a CSK protein kinase selected from the group consisting of SEQ ID NO:8 and SEQ ID NO:9 or a subsequence thereof, with the proviso that any two amino acids in the sequence AA₁ through AA₁₈ or the subsequence thereof can vary according to the listing in claim 30.

32. The peptide of claim 30 wherein the sequence AA₁ through AA₁₈ or a subsequence thereof corresponds to a sequence of the A region of a CSK protein kinase selected from the group consisting of SEQ ID NO:8 and SEQ ID NO:9 or a subsequence thereof, with the proviso that any one amino acid in the sequence AA₁ through AA₁₈ or the subsequence thereof can vary according to the listing in claim 30.

33. A peptide comprising a sequence of amino acids AA₁ through AA₁₉ or a subsequence thereof comprising at least five amino acids, wherein:
AA₁ is selected from the group consisting of leucine, isoleucine, methionine and valine;
AA₂ is selected from the group consisting of glutamic acid, aspartic acid, glutamine, asparagine and an aliphatic, substituted aliphatic, benzyl, substituted benzyl, aromatic or substituted aromatic ester of a glutamic or aspartic acid;
AA₃ is selected from the group consisting of valine, isoleucine, leucine and methionine;
AA₄ is selected from the group consisting of leucine, isoleucine, methionine and valine;
AA₅ is selected from the group consisting of cysteine and serine;
AA₆ is selected from the group consisting of lysine and arginine;
AA₇ is selected from the group consisting of leucine, isoleucine, methionine and valine;
AA₈ is selected from the group consisting of glycine and alanine;
AA₉ is histidine;
AA₁₀ is histidine;
AA₁₁ is proline;
AA₁₂ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₃ is selected from the group consisting of isoleucine, leucine, methionine and valine;
AA₁₄ is selected from the group consisting of isoleucine, leucine, methionine and valine;
AA₁₅ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₆ is selected from the group consisting of leucine, isoleucine, methionine and valine;
AA₁₇ is selected from the group consisting of leucine, isoleucine, methionine and valine;
AA₁₈ is selected from the group consisting of glycine and alanine; and
AA₁₉ is selected from the group consisting of alanine and glycine.

34. The peptide of claim 33 wherein the sequence AA₁ through AA₁₉ or a subsequence thereof corresponds to the sequence of the A region of an endothelial protein kinase designated as SEQ ID NO:12 or a subsequence thereof, with the proviso that any two amino acids in the sequence AA₁ through AA₁₉ or the subsequence thereof can vary according to the listing in claim 33.

35. The peptide of claim 33 wherein the sequence AA₁ through AA₁₉ or a subsequence thereof corresponds to the sequence of the A region of an endothelial protein kinase designated as SEQ ID NO:12 or a subsequence thereof, with the proviso that any one amino acid in the sequence AA₁ through AA₁₉ or the subsequence thereof can vary according to the listing in claim 33.

36. A peptide comprising a sequence of amino acids AA₁ through AA₁₉ or a subsequence thereof comprising at least five amino acids, wherein:
AA₁ is selected from the group consisting of methionine, isoleucine, leucine, and valine;
AA₂ is selected from the group consisting of glutamic acid, glutamine, aspartic acid, asparagine and an aliphatic, substituted aliphatic, benzyl, substituted benzyl, aromatic or substituted aromatic ester of a glutamic or aspartic acid;
AA₃ is selected from the group consisting of valine, isoleucine, leucine and methionine;
AA₄ is selected from the group consisting of methionine, leucine, isoleucine and valine;
AA₅ is selected from the group consisting of lysine and arginine;
AA₆ is selected from the group consisting of methionine, leucine, isoleucine and valine;
AA₇ is selected from the group consisting of isoleucine, leucine, methionine and valine;
AA₈ is selected from the group consisting of glycine and alanine;
AA₉ is selected from the group consisting of lysine and arginine;
AA₁₀ is histidine;
AA₁₁ is selected from the group consisting of lysine and arginine;
AA₁₂ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₃ is selected from the group consisting of isoleucine, leucine, methionine and valine;
AA₁₄ is selected from the group consisting of isoleucine, leucine, methionine and valine;
AA₁₅ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₆ is selected from the group consisting of leucine, isoleucine, methionine and valine;
AA₁₇ is selected from the group consisting of leucine, isoleucine, methionine and valine;
AA₁₈ is selected from the group consisting of glycine and alanine; and
AA₁₉ is selected from the group consisting of alanine and glycine.

37. The peptide of claim 36 wherein the sequence AA₁ through AA₁₉ or a subsequence thereof corresponds to a sequence of the A region of an endothelial protein kinase selected from the group consisting of SEQ ID NO: 13 and SEQ ID NO: 14 or a subsequence thereof, with the proviso that any two amino acids in the sequence AA₁ through AA₁₉ or the subsequence thereof can vary according to the listing in claim 36.

38. The peptide of claim 36 wherein the sequence AA₁ through AA₁₉ or a subsequence thereof corresponds to a sequence of the A region of an endothelial protein kinase selected from the group consisting of SEQ ID NO: 13 and SEQ ID NO:14 or a subsequence thereof, with the proviso that any one amino acid in the sequence AA₁ through AA₁₉ or the subsequence thereof can vary according to the listing in claim 36.

39. A peptide comprising a sequence of amino acids AA₁ through AA₁₉ or a subsequence thereof comprising at least five amino acids, wherein:
AA₁ is selected from the group consisting of leucine, isoleucine, methionine and valine;
AA₂ is selected from the group consisting of lysine and arginine;
AA₃ is selected from the group consisting of isoleucine, leucine, methionine and valine;
AA₄ is selected from the group consisting of methionine, leucine, isoleucine and valine;
AA₅ is selected from the group consisting of threonine, serine, isoleucine, leucine, methionine and valine;
AA₆ is histidine;
AA₇ is selected from the group consisting of leucine, isoleucine, methionine and valine;
AA₈ is selected from the group consisting of glycine and alanine;
AA₉ is selected from the group consisting of proline, histidine, asparagine, glutamine, glutamic acid, aspartic acid and an aliphatic, substituted aliphatic, benzyl, substituted benzyl, aromatic or substituted aromatic ester of a glutamic or aspartic acid;
AA₁₀ is histidine;
AA₁₁ is selected from the group consisting of leucine, isoleucine, methionine an valine;
AA₁₂ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₃ is selected from the group consisting of isoleucine, valine, leucine and methionine;
AA₁₄ is selected from the group consisting of valine, isoleucine, leucine and methionine;
AA₁₅ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₆ is selected from the group consisting of leucine, isoleucine, methionine and valine;
AA₁₇ is selected from the group consisting of leucine, isoleucine, methionine and valine;
AA₁₈ is selected from the group consisting of glycine and alanine; and
AA₁₉ is selected from the group consisting of alanine and glycine.

40. The peptide of claim 39 wherein the sequence AA₁ through AA₁₉ or a subsequence thereof corresponds to a sequence of the A region of an endothelial protein kinase selected from the group consisting of SEQ ID NO: 15, SEQ ID NO: 16, SEQ ID NO: 17, SEQ ID NO: 18, and SEQ ID NO: 19 or a subsequence thereof, with the proviso that any two amino acids in the sequence AA₁ through AA₁₉ or the subsequence thereof can vary according to the listing in claim 39.

41. The peptide of claim 39 wherein the sequence AA₁ through AA₁₉ or a subsequence thereof corresponds to a sequence of the A region of an endothelial protein kinase selected from the group consisting of SEQ ID NO: 15, SEQ ID NO:16, SEQ ID NO:17, SEQ ID NO:18, and SEQ ID NO:19 or a subsequence thereof, with the proviso that any one amino acid in the sequence AA₁ through AA₁₉ or the subsequence thereof can vary according to the listing in claim 39.

42. A peptide comprising a sequence of amino acids AA₁ through AA₁₈ or a subsequence thereof comprising at least five amino acids, wherein:
AA₁ is selected from the group consisting of glycine and alanine;
AA₂ is selected from the group consisting of isoleucine, leucine, methionine and valine;
AA₃ is selected from the group consisting of isoleucine, leucine, methionine and valine;
AA₄ is selected from the group consisting of methionine, isoleucine, leucine and valine;
AA₅ is selected from the group consisting of lysine and arginine;
AA₆ is selected from the group consisting of aspartic acid, serine, glycine, glutamic acid, glutamine, asparagine, threonine, alanine and an aliphatic, substituted aliphatic, benzyl, substituted benzyl, aromatic or substituted aromatic ester of a glutamic or aspartic acid;
AA₇ is selected from the group consisting of phenylalanine, leucine, tryptophan, tyrosine, isoleucine, methionine and valine;
AA₈ is selected from the group consisting of serine, histidine, asparagine, threonine, glutamic acid, aspartic acid, glutamine and an aliphatic, substituted aliphatic, benzyl, substituted benzyl, aromatic or substituted aromatic ester of a glutamic or aspartic acid;
AA₉ is histidine;
AA₁₀ is proline;
AA₁₁ is selected from the group consisting of asparagine, glutamine, glutamic acid, aspartic acid, and an aliphatic, substituted aliphatic, benzyl, substituted benzyl, aromatic or substituted aromatic ester of a glutamic or aspartic acid;
AA₁₂ is selected from the group consisting of valine, isoleucine, leucine and methionine;
AA₁₃ is selected from the group consisting of leucine, isoleucine, methionine and valine;
AA₁₄ is selected from the group consisting of serine, alanine, threonine and glycine;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine and alanine; and
AA₁₈ is selected from the group consisting of isoleucine, valine, leucine and methionine.

43. The peptide of claim 42 wherein the sequence AA₁ through AA₁₈ or a subsequence thereof corresponds to a sequence of the A region of an HGF receptor protein kinase selected from the group consisting of SEQ ID NO:20, SEQ ID NO:21 and SEQ ID NO:22 or a subsequence thereof, with the proviso that any two amino acids in the sequence AA₁ through AA₁₈ or the subsequence thereof can vary according to the listing in claim 42.

44. The peptide of claim 42 wherein the sequence AA₁ through AA₁₈ or a subsequence thereof corresponds to a sequence of the A region of an HGH receptor protein kinase selected from the group consisting of SEQ ID NO:20, SEQ ID NO:21 and SEQ ID NO:22 or a subsequence thereof, with the proviso that any one amino acid in the sequence AA₁ through AA₁₈ or the subsequence thereof can vary according to the listing in claim 42.

45. A peptide comprising a sequence of amino acids AA₁ through AA₁₈ or a subsequence thereof comprising at least five amino acids, wherein:
AA₁ is selected from the group consisting of alanine, methionine, glycine, isoleucine, leucine and valine;
AA₂ is selected from the group consisting of tyrosine, leucine, phenylalanine, tryptophan, isoleucine, methionine and valine;
AA₃ is selected from the group consisting of valine, alanine, isoleucine, leucine, methionine and glycine;
AA₄ is selected from the group consisting of methionine, isoleucine, leucine and valine;
AA₅ is selected from the group consisting of alanine and glycine;
AA₆ is selected from the group consisting of serine, glycine, threonine and alanine;
AA₇ is selected from the group consisting of valine, leucine, methionine and isoleucine;
AA₈ is selected from the group consisting of aspartic acid, glycine, glutamic acid, glutamine, asparagine, alanine and an aliphatic, substituted aliphatic, benzyl, substituted benzyl, aromatic or substituted aromatic ester of a glutamic or aspartic acid;
AA₉ is selected from the group consisting of asparagine, serine, histidine, glutamic acid, aspartic acid, glutamine, threonine and an aliphatic, substituted aliphatic, benzyl, substituted benzyl, aromatic or substituted aromatic ester of a glutamic or aspartic acid;
AA₁₀ is selected from the group consisting of proline, alanine and glycine;
AA₁₁ is selected from the group consisting of histidine, tyrosine, phenylalanine and tryptophan;
AA₁₂ is selected from the group consisting of valine, isoleucine, leucine and methionine;
AA₁₃ is selected from the group consisting of cysteine, serine, valine, isoleucine, leucine and methionine;
AA₁₄ is selected from the group consisting of arginine and lysine;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine and alanine; and
AA₁₈ is selected from the group consisting of isoleucine, leucine, valine, and methionine.

46. The peptide of claim 45 wherein the sequence AA₁ through AA₁₈ or a subsequence thereof corresponds to a sequence of the A region of an EGF receptor protein kinase selected from the group consisting of SEQ ID NO:23, SEQ ID NO:24, SEQ ID NO:25 and SEQ ID NO:26 or a subsequence thereof, with the proviso that any two amino acids in the sequence AA₁ through AA₁₈ or the subsequence thereof can vary according to the listing in claim 45.

47. The peptide of claim 45 wherein the sequence AA₁ through AA₁₈ or a subsequence thereof corresponds to a sequence of the A region of an EGF receptor protein kinase selected from the group consisting of SEQ ID NO:23, SEQ ID NO:24, SEQ ID NO:25 and SEQ ID NO:26 or a subsequence thereof, with the proviso that any one amino acid in the sequence AA₁ through AA₁₈ or the subsequence thereof can vary according to the listing in claim 45.

48. A peptide comprising SEQ ID NO.: 27 or a subsequence thereof comprising at least five amino acids, wherein the amino acid sequence of said peptide corresponds to a sequence of the A region of a Ret receptor protein kinase.

49. A peptide comprising a sequence of amino acids AA₁ through AA₁₈ or a subsequence thereof comprising at least five amino acids, wherein:
AA₁ is selected from the group consisting of valine, alanine, isoleucine, leucine, methionine and glycine;
AA₂ is selected from the group consisting of glutamic acid, aspartic acid, glutamine, asparagine and an aliphatic, substituted aliphatic, benzyl, substituted benzyl, aromatic or substituted aromatic ester of a glutamic or aspartic acid;
AA₃ is selected from the group consisting of leucine, isoleucine, methionine and valine;
AA₄ is selected from the group consisting of leucine, isoleucine, methionine and valine;
AA₅ is selected from the group consisting of threonine and serine;
AA₆ is selected from the group consisting of methionine, asparagine, isoleucine, leucine, valine, glutamic acid, aspartic acid, glutamine and an aliphatic, substituted aliphatic, benzyl, substituted benzyl, aromatic or substituted aromatic ester of a glutamic or aspartic acid;
AA₇ is selected from the group consisting of leucine, isoleucine, methionine and valine;
AA₈ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₉ is histidine;
AA₁₀ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₁₁ is histidine;
AA₁₂ is selected from the group consisting of isoleucine, leucine, methionine and valine;
AA₁₃ is selected from the group consisting of valine, isoleucine, leucine and methionine;
AA₁₄ is selected from the group consisting of arginine and lysine;
AA₁₅ is selected from the group consisting of phenylalanine, tryptophan and tyrosine;
AA₁₆ is selected from the group consisting of phenylalanine, tyrosine and tryptophan;
AA₁₇ is selected from the group consisting of glycine and alanine; and
AA₁₈ is selected from the group consisting of valine, isoleucine, leucine and methionine.

50. The peptide of claim 49 wherein the sequence AA₁ through AA₁₈ or a subsequence thereof corresponds to a sequence of the A region of an NGF receptor protein kinase selected from the group consisting of SEQ ID NO:28, SEQ ID NO:29 and SEQ ID NO:30 or a subsequence thereof, with the proviso that any two amino acids in the sequence AA₁ through AA₁₈ or the subsequence thereof can vary according to the listing in claim 49.

51. The peptide of claim 49 wherein the sequence AA₁ through AA₁₈ or a subsequence thereof corresponds to a sequence of the A region of an NGF receptor protein kinase selected from the group consisting of SEQ ID NO:28, SEQ ID NO:29 and SEQ ID NO:30 or a subsequence thereof, with the proviso that any one amino acid in the sequence AA₁ through AA₁₈ or the subsequence thereof can vary according to the listing in claim 49.

52. A peptide comprising a sequence of amino acids AA₁ through AA₁₈ or a subsequence thereof comprising at least five amino acids, wherein:
AA₁ is selected from the group consisting of alanine and glycine;
AA₂ is selected from the group consisting of asparagine, glutamine, glutamic acid, and aspartic acid;
AA₃ is selected from the group consisting of valine, isoleucine, leucine and methionine;
AA₄ is selected from the group consisting of methionine, isoleucine, leucine and valine;
AA₅ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₆ is selected from the group consisting of glutamine, glutamic acid, aspartic acid, asparagine and an aliphatic, substituted aliphatic, benzyl, substituted benzyl, aromatic or substituted aromatic ester of a glutamic or aspartic acid;
AA₇ is selected from the group consisting of leucine, isoleucine, methionine and valine;
AA₈ is selected from the group consisting of aspartic acid, glutamic acid, glutamine, asparagine and an aliphatic, substituted aliphatic, benzyl, substituted benzyl, aromatic or substituted aromatic ester of a glutamic or aspartic acid;
AA₉ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₀ is proline;
AA₁₁ is selected from the group consisting of tyrosine, phenylalanine and tryptophan;
AA₁₂ is selected from the group consisting of isoleucine, leucine, methionine and valine;
AA₁₃ is selected from the group consisting of valine, isoleucine, leucine and methionine;
AA₁₄ is selected from the group consisting of arginine and lysine;
AA₁₅ is selected from the group consisting of methionine, leucine, isoleucine and valine;
AA₁₆ is selected from the group consisting of isoleucine, leucine, methionine and valine;
AA₁₇ is selected from the group consisting of glycine and alanine; and
AA₁₈ is selected from the group consisting of isoleucine, valine, leucine and methionine.

53. The peptide of claim 52 wherein the sequence AA₁ through AA₁₈ or a subsequence thereof corresponds to a sequence of the A region of a Syk or Zap70 protein kinase selected from the group consisting of SEQ ID NO:30 and SEQ ID NO:31 or a subsequence thereof, with the proviso that any two amino acids in the sequence AA₁ through AA₁₈ or the subsequence thereof can vary according to the listing in claim 52.

54. The peptide of claim 52 wherein the sequence AA₁ through AA₁₈ or a subsequence thereof corresponds to a sequence of the A region of an Syk or Zap70 protein kinase selected from the group consisting of SEQ ID NO:30 and SEQ ID NO:31 or a subsequence thereof, with the proviso that any one amino acid in the sequence AA₁ through AA₁₈ or the subsequence thereof can vary according to the listing in claim 52.

55. A peptide comprising a sequence of amino acids AA₁ through AA₁₈ or a subsequence thereof comprising at least five amino acids, wherein:
AA₁ is selected from the group consisting of isoleucine, leucine, methionine and valine;
AA₂ is selected from the group consisting of glutamic acid, glutamine, aspartic acid, asparagine and an aliphatic, substituted aliphatic, benzyl, substituted benzyl, aromatic or substituted aromatic ester of a glutamic or aspartic acid;
AA₃ is selected from the group consisting of isoleucine, leucine, methionine and valine;
AA₄ is selected from the group consisting of leucine, isoleucine, methionine and valine;
AA₅ is selected from the group consisting of arginine and lysine;
AA₆ is selected from the group consisting of asparagine, serine, alanine, threonine, glutamine, glutamic acid, aspartic acid, glycine and an aliphatic, substituted aliphatic, benzyl, substituted benzyl, aromatic or substituted aromatic ester of a glutamic or aspartic acid;
AA₇ is selected from the group consisting of leucine, isoleucine, methionine and valine;
AA₈ is selected from the group consisting of tyrosine, glutamine, histidine, phenylalanine, tryptophan, glutamic acid, aspartic acid, asparagine and an aliphatic, substituted aliphatic, benzyl, substituted benzyl, aromatic or substituted aromatic ester of a glutamic or aspartic acid;
AA₉ is selected from the group consisting of histidine, serine and threonine; AA₁₀ is selected from the group consisting of glutamic acid, aspartic acid, asparagine, glutamine and an aliphatic, substituted aliphatic, benzyl, substituted benzyl, aromatic or substituted aromatic ester of a glutamic or aspartic acid;
AA₁₁ is selected from the group consisting of asparagine, phenylalanine, histidine, glutamic acid, aspartic acid, glutamine, tryptophan, tyrosine and an aliphatic, substituted aliphatic, benzyl, substituted benzyl, aromatic or substituted aromatic ester of a glutamic or aspartic acid;
AA₁₂ is selected from the group consisting of isoleucine, leucine, methionine and valine;
AA₁₃ is selected from the group consisting of valine, isoleucine, leucine and methionine;
AA₁₄ is selected from the group consisting of lysine and arginine;
AA₁₅ is selected from the group consisting of tyrosine, phenylalanine, and tryptophan;
AA₁₆ is selected from the group consisting of lysine and arginine;
AA₁₇ is selected from the group consisting of glycine and alanine; and
AA₁₈ is selected from the group consisting of isoleucine, valine, cysteine, leucine, methionine and serine.

56. The peptide of claim 55 wherein the sequence AA₁ through AA₁₈ or a subsequence thereof corresponds to a sequence of the A region of a Jak or Tyk protein kinase selected from the group consisting of SEQ ID NO:32, SEQ ID NO:33, SEQ ID NO:34, and SEQ ID NO:35 or a subsequence thereof, with the proviso that any two amino acids in the sequence AA₁ through AA₁₈ or the subsequence thereof can vary according to the listing in claim 55.

57. The peptide of claim 55 wherein the sequence AA₁ through AA₁₈ or a subsequence thereof corresponds to a sequence of the A region of a Jak or Tyk protein kinase selected from the group consisting of SEQ ID NO:32, SEQ ID NO:33, SEQ ID NO:34, and SEQ ID NO:35 or a subsequence thereof, with the proviso that any one amino acid in the sequence AA₁ through AA₁₈ or the subsequence thereof can vary according to the listing in claim 55.

58. A peptide comprising SEQ ID NO.:36 or a subsequence thereof comprising at least five amino acids, wherein the amino acid sequence of said peptide corresponds to a sequence of the A region of an IRK receptor protin kinase.

59. A peptide comprising a sequence of amino acids AA₁ through AA₁₈ or a subsequence thereof comprising at least five amino acids, wherein:
AA₁ is selected from the group consisting of isoleucine, leucine, methionine and valine;
AA₂ is selected from the group consisting of tyrosine, phenylalanine and tryptophan;
AA₃ is selected from the group consisting of asparagine, glutamine, glutamic acid, aspartic acid and an aliphatic, substituted aliphatic, benzyl, substituted benzyl, aromatic or substituted aromatic ester of a glutamic or aspartic acid;
AA₄ is selected from the group consisting of threonine and serine;
AA₅ is selected from the group consisting of valine, isoleucine, leucine and methionine;
AA₆ is selected from the group consisting of leucine, methionine, isoleucine, and valine;
AA₇ is selected from the group consisting of leucine, methionine, isoleucine and valine;
AA₈ is selected from the group consisting of arginine and lysine;
AA₉ is histidine;
AA₁₀ is selected from the group consisting of aspartic acid, glutamic acid, asparagine, glutamine and an aliphatic, substituted aliphatic, benzyl, substituted benzyl, aromatic or substituted aromatic ester of a glutamic or aspartic acid;
AA₁₁ is selected from the group consisting of asparagine, glutamine, glutamic acid, aspartic acid and an aliphatic, substituted aliphatic, benzyl, substituted benzyl, aromatic or substituted aromatic ester of a glutamic or aspartic acid;
AA₁₂ is selected from the group consisting of isoleucine, leucine, methionine and valine;
AA₁₃ is selected from the group consisting of leucine, isoleucine, methionine and valine;
AA₁₄ is selected from the group consisting of glycine and alanine;
AA₁₅ is selected from the group consisting of phenylalanine, tryptophan and tyrosine;
AA₁₆ is selected from the group consisting of isoleucine, leucine, methionine and valine;
AA₁₇ is selected from the group consisting of alanine and glycine; and
AA₁₈ is selected from the group consisting of serine, alanine, glycine and threonine.

60. The peptide of claim 59 wherein the sequence AA₁ through AA₁₈ or a subsequence thereof corresponds to a sequence of the A region of an activin receptor-like protein kinase selected from the group consisting of SEQ ID NO: 37, SEQ ID NO: 38, SEQ ID NO: 39 and SEQ ID NO: 40 or a subsequence thereof, with the proviso that any two amino acids in the sequence AA₁ through AA₁₈ or the subsequence thereof can vary according to the listing in claim 59.

61. The peptide of claim 59 wherein the sequence AA₁ through AA₁₈ or a subsequence thereof corresponds to a sequence of the A region of an activin receptor-like protein kinase selected from the group consisting of SEQ ID NO: 37, SEQ ID NO: 38, SEQ ID NO: 39 and SEQ ID NO: 40 or a subsequence thereof, with the proviso that any one amino acid in the sequence AA₁ through AA₁₈ or the subsequence thereof can vary according to the listing in claim 59.

62. A peptide comprising a sequence of amino acids AA₁ through AA₁₈ or a subsequence thereof comprising at least five amino acids, wherein:
AA₁ is selected from the group consisting of valine, isoleucine, leucine and methionine;
AA₂ is selected from the group consisting of lysine and arginine;
AA₃ is selected from the group consisting of isoleucine, leucine, methionine and valine;
AA₄ is selected from the group consisting of methionine, isoleucine, leucine and valine;
AA₅ is selected from the group consisting of serine and threonine;
AA₆ is selected from the group consisting of arginine and lysine;
AA₇ is selected from the group consisting of leucine, isoleucine, methionine and valine;
AA₈ is selected from the group consisting of lysine and arginine;
AA₉ is selected from the group consisting of aspartic acid, glutamic acid, asparagine, glutamine and an aliphatic, substituted aliphatic, benzyl, substituted benzyl, aromatic or substituted aromatic ester of a glutamic or aspartic acid;
AA₁₀ is proline;
AA₁₁ is selected from the group consisting of asparagine, glutamic acid, aspartic acid, glutamine and an aliphatic, substituted aliphatic, benzyl, substituted benzyl, aromatic or substituted aromatic ester of a glutamic or aspartic acid;
AA₁₂ is selected from the group consisting of isoleucine, leucine, methionine and valine;
AA₁₃ is selected from the group consisting of isoleucine, leucine, methionine and valine;
AA₁₄ is selected from the group consisting of arginine, histidine and lysine;
AA₁₅ is selected from the group consisting of leucine, isoleucine, methionine and valine;
AA₁₆ is selected from the group consisting of leucine, isoleucine, methionine and valine;
AA₁₇ is selected from the group consisting of glycine, serine, alanine and threonine; and
AA₁₈ is selected from the group consisting of valine, isoleucine, leucine and methionine.

63. The peptide of claim 62 wherein the sequence AA₁ through AA₁₈ or a subsequence thereof corresponds to a sequence of the A region of a discoidin domain receptor protein kinase selected from the group consisting of SEQ ID NO: 41 and SEQ ID NO: 42 or a subsequence thereof, with the proviso that any two amino acids in the sequence AA₁ through AA₁₈ or the subsequence thereof can vary according to the listing in claim 62.

64. The peptide of claim 62 wherein the sequence AA₁ through AA₁₈ or a subsequence thereof corresponds to a sequence of the A region of a discoidin domain receptor protein kinase selected from the group consisting of SEQ ID NO: 41 and SEQ ID NO: 42 or a subsequence thereof, with the proviso that any one amino acid in the sequence AA₁ through AA₁₈ or the subsequence thereof can vary according to the listing in claim 62.

65. A method of identifying a peptide which modulates the activity of a protein kinase comprising the steps of:
a) providing a peptide, referred to as a “test peptide”, consisting of a peptide derivative of the A region of said protein kinase which has from about five to about twenty amino acids, conservatively substituted amino acids or functional peptidomimetics thereof;
b) incubating the peptide with cells having one or more cellular activities controlled by a protein kinase under conditions suitable for assessing activity of the protein kinase;
c) assessing activity of the protein kinase, wherein greater or lesser activity compared with the cells grown without incubation of the test peptide indicates that the peptide modulates activity of the protein kinase.

66. The method of claim 65, wherein the activity of the protein kinase is assessed by measuring the rate of survival or proliferation of said cells in tissue culture.

67. A method of modulating the activity of a protein kinase in a subject, comprising administering a therapeutically effective amount of a peptide comprising a peptide derivative of the A region of a protein kinase, wherein:
a) said peptide has between about five and about twenty amino acids, conservatively substituted amino acids or functional peptidomimetics thereof; and
b) said peptide modulates activity of the protein kinase.

68. A method of detecting a ligand that binds to the A region of a protein kinase comprising:
a) providing a peptide derivative of the A region of said protein kinase, said peptide derivative having at least five amino acids, conservatively substituted amino acid or functional peptidomimetics thereof;
b) incubating said peptide derivative with a sample, to be tested for the presence of said ligand, for a time sufficient for said ligand to bind to said peptide derivative; and
c) detecting any said ligand-said peptide derivative binding pair that has been formed in step b), wherein the presence of said ligand-said peptide derivative binding pair establishes the existence of said ligand in said sample.

69. The method of claim 68 further comprising:
d) separating said ligand from said peptide derivative; and
e) determining the structure of said ligand, thereby identifying said ligand.

70. An antibody that immunologically binds to the A region of a protein kinase.

71. A method of producing antibodies that bind to the A region of a protein kinase comprising:
a) providing a peptide derivative of the A region of said protein kinase, said peptide derivative having at least five amino acids; and
b) producing antibodies to said peptide derivatives.


SEQUENCE LISTING (122 sequences)

SEQ ID NO: 1 - protein, 18 aa, Unknown
  c-Src
AQVMKKLRHEKLVQLYAV

SEQ ID NO: 2 - protein, 18 aa, Unknown
  c-Yes
AQIMKKLRHDKLVPLYAV

SEQ ID NO: 3 - protein, 18 aa, Unknown
  Fyn
AQIMKKLKHDKLVQLYAV

SEQ ID NO: 4 - protein, 18 aa, Unknown
  c-Fgr
AQVMKLLRHDKLVQLYAV

SEQ ID NO: 5 - protein, 18 aa, Unknown
  Lyn
ANLMKTLQHDKLVRLYAV

SEQ ID NO: 6 - protein, 18 aa, Unknown
  Hck
ANVMKTLQHDKLVKLHAV

SEQ ID NO: 7 - protein, 18 aa, Unknown
  Lck
ANLMKQLQHQRLVRLYAV

SEQ ID NO: 8 - protein, 18 aa, Unknown
  Csk
ASVMTQLRHSNLVQLLGV

SEQ ID NO: 9 - protein, 18 aa, Unknown
  Matk
TAVMTKMQHENLVRLLGV

SEQ ID NO: 10 - protein, 18 aa, Unknown
  Fak
ALTMRQFDHPHIVKLIGV

SEQ ID NO: 11 - protein, 18 aa, Unknown
  c-Abl
AAVMKEIKHPNLVQLLGV

SEQ ID NO: 12 - protein, 19 aa, Unknown
  Tie/Tek
LEVLCKLGHHPNIINLLGA

SEQ ID NO: 13 - protein, 19 aa, Unknown
  FGFR
MEMMKMIGKHKNIINLLGA

SEQ ID NO: 14 - protein, 19 aa, Unknown
  FGFR
MEVMKLIGRHKNIINLLGV

SEQ ID NO: 15 - protein, 19 aa, Unknown
  PDGFR-a
LKIMTHLGPHLNIVNLLGA

SEQ ID NO: 16 - protein, 19 aa, Unknown
  PDGFR-b
LKIMSHLGPHLNVVNLLGA

SEQ ID NO: 17 - protein, 19 aa, Unknown
  Flt1
LKILTHIGHHLNVVNLLGA

SEQ ID NO: 18 - protein, 19 aa, Unknown
  Flt4
LKILIHIGNHLNVVNLLGA

SEQ ID NO: 19 - protein, 19 aa, Unknown
  Flk1
LKILIHIGHHLNVVNLLGA

SEQ ID NO: 20 - protein, 18 aa, Unknown
  c-Met
GIIMKDFSHPNVLSLLGI

SEQ ID NO: 21 - protein, 18 aa, Unknown
  c-Sea
GILMKSFHHPQVLSLLGV

SEQ ID NO: 22 - protein, 18 aa, Unknown
  Ron
GLLMRGLNHPNVLALIGI

SEQ ID NO: 23 - protein, 18 aa, Unknown
  EGFR
AYVMASVDNPHVCRLLGI

SEQ ID NO: 24 - protein, 18 aa, Unknown
  ErbB2
AYVMAGVGSPYVSRLLGI

SEQ ID NO: 25 - protein, 18 aa, Unknown
  ErbB3
MLAIGSLDHAHIVRLLGL

SEQ ID NO: 26 - protein, 18 aa, Unknown
  ErbB4
ALIMASMDHPHLVRLLGV

SEQ ID NO: 27 - protein, 18 aa, Unknown
  Ret
FNVLKQVNHPHVIKLYGA

SEQ ID NO: 28 - protein, 18 aa, Unknown
  Trk-NGFR
VELLTMLQHQHIVRFFGV

SEQ ID NO: 29 - protein, 18 aa, Unknown
  TrkB/TrkC
AELLTNLQHEHIVKFYGV

SEQ ID NO: 30 - protein, 18 aa, Unknown
  Syk
ANVMQQLDNPYIVRMIGI

SEQ ID NO: 31 - protein, 18 aa, Unknown
  Zap70
AQIMEQLDNPYIVRLIGV

SEQ ID NO: 32 - protein, 18 aa, Unknown
  Jak1
IEILRNLYHENIVKYKGI

SEQ ID NO: 33 - protein, 18 aa, Unknown
  Jak2
IEILKSLQHDNIVKYKGV

SEQ ID NO: 34 - protein, 18 aa, Unknown
  Jak3
IQILKALHSDFIVKYRGV

SEQ ID NO: 35 - protein, 18 aa, Unknown
  Tyk2
IDILRTLYHEHIIKYKGC

SEQ ID NO: 36 - protein, 18 aa, Unknown
  IRK
ASVMKGFTCHHVVRLLGV

SEQ ID NO: 37 - protein, 18 aa, Unknown
  ALK1
IYNTVLLRHDNILGFIAS

SEQ ID NO: 38 - protein, 18 aa, Unknown
  ALK2
LYNTVMLRHENILGFIAS

SEQ ID NO: 39 - protein, 18 aa, Unknown
  ALK3/ALK6
IYQTVLMRHENILGFIAA

SEQ ID NO: 40 - protein, 18 aa, Unknown
  ALK4/ALK5
IYQTVMLRHENILGFIAA

SEQ ID NO: 41 - protein, 18 aa, Unknown
  DDR1
VKIMSRLKDPNIIRLLGV

SEQ ID NO: 42 - protein, 18 aa, Unknown
  DDR2
IKIMSRLKDPNIIHLLSV

SEQ ID NO: 43 - protein, 18 aa, Unknown
  ACK
VNAMHSLDHRNLIRLYGV

SEQ ID NO: 44 - protein, 18 aa, Unknown
  Eph-B4
ASIMGQFEHPNIIRLEGV

SEQ ID NO: 45 - protein, 18 aa, Unknown
  ITK/TSK
AEVMMKLSHPKLVQLYGV

SEQ ID NO: 46 - protein, 18 aa, Unknown
  Plk
ISIHRSLAHQHVVGFHGF

SEQ ID NO: 47 - protein, 18 aa, Unknown
  Plx1
IEILATCNHHFIVKLLGA

SEQ ID NO: 48 - protein, 18 aa, Unknown
  Polo
ITIHRSLNHPNIVKFHNY

SEQ ID NO: 49 - protein, 18 aa, Unknown
  SNK
IELHRILHHKHVVQFYHY

SEQ ID NO: 50 - protein, 18 aa, Unknown
  CDC5
IQIHKSMSHPNIVQFIDC

SEQ ID NO: 51 - protein, 18 aa, Unknown
  Sak
VKIHCQLKHPSVLELYNY

SEQ ID NO: 52 - protein, 18 aa, Unknown
  Prk/Fnk
IELHRDLQHRHIVRFSHH

SEQ ID NO: 53 - protein, 18 aa, Unknown
  Plo1
IKVHQSMSHPNIVGFIDC

SEQ ID NO: 54 - protein, 11 aa, Artificial Sequence
  plk
GSLAHQHVVGF

SEQ ID NO: 55 - protein, 11 aa, Artificial Sequence
  plx1
GTCNHHFIVKL

SEQ ID NO: 56 - protein, 11 aa, Artificial Sequence
  polo
GSLNHPNIVKF

SEQ ID NO: 57 - protein, 11 aa, Artificial Sequence
  Snk
GILHHKHVVQF

SEQ ID NO: 58 - protein, 11 aa, Artificial Sequence
  CDC5
GSMSHPNIVQF

SEQ ID NO: 59 - protein, 11 aa, Artificial Sequence
  Sak
GQLKHPSVLEL

SEQ ID NO: 60 - protein, 11 aa, Artificial Sequence
  Prk
GDLQHRHIVRF

SEQ ID NO: 61 - protein, 11 aa, Artificial Sequence
  plo1
GSMSHPNIVGF

SEQ ID NO: 62 - protein, 11 aa, Artificial Sequence
  Alk1
GLLRHDNILGF

SEQ ID NO: 63 - protein, 11 aa, Artificial Sequence
  c-Src
GKLRHEKLVQL

SEQ ID NO: 64 - protein, 11 aa, Artificial Sequence
  c-Yes
GKLRHDKLVPL

SEQ ID NO: 65 - protein, 11 aa, Artificial Sequence
  Fyn
GKLKHDKLVQL

SEQ ID NO: 66 - protein, 11 aa, Artificial Sequence
  c-Fgr
GLLRHDKLVQL

SEQ ID NO: 67 - protein, 11 aa, Artificial Sequence
  Lyn
GTLQHDKLVRL

SEQ ID NO: 68 - protein, 11 aa, Artificial Sequence
  Hck
GTLQHDKLVKL

SEQ ID NO: 69 - protein, 11 aa, Artificial Sequence
  Lck
GQLQHQRLVRL

SEQ ID NO: 70 - protein, 11 aa, Artificial Sequence
  Csk
GQLRHSNLVQL

SEQ ID NO: 71 - protein, 11 aa, Artificial Sequence
  Matk
GKMQHENLVRL

SEQ ID NO: 72 - protein, 11 aa, Artificial Sequence
  Fak
GQFDHPHIVKL

SEQ ID NO: 73 - protein, 11 aa, Artificial Sequence
  c-Abl
GEIKHPNLVQL

SEQ ID NO: 74 - protein, 12 aa, Artificial Sequence
  Tie
GKLGHNPNIINL

SEQ ID NO: 75 - protein, 12 aa, Artificial Sequence
  PDGFR-b
GHLGPHLNVVNL

SEQ ID NO: 76 - protein, 12 aa, Artificial Sequence
  PDGFR-a
GHLGPHLNIVNL

SEQ ID NO: 77 - protein, 12 aa, Artificial Sequence
  Flt1
GHIGHHLNVVNL

SEQ ID NO: 78 - protein, 12 aa, Artificial Sequence
  Flt4
GHIGNHLNVVNL

SEQ ID NO: 79 - protein, 12 aa, Artificial Sequence
  Flg
GMIGKHKNIINL

SEQ ID NO: 80 - protein, 12 aa, Artificial Sequence
  FGFR-4
GLIGRHKNIINL

SEQ ID NO: 81 - protein, 11 aa, Artificial Sequence
  c-Met
GDFSHPNVLSL

SEQ ID NO: 82 - protein, 11 aa, Artificial Sequence
  c-Sea
GSFHHPQVLSL

SEQ ID NO: 83 - protein, 11 aa, Artificial Sequence
  Ron
GGLNHPNVLAL

SEQ ID NO: 84 - protein, 11 aa, Artificial Sequence
  EGFR
GSVDNPHVCRL

SEQ ID NO: 85 - protein, 11 aa, Artificial Sequence
  ErbB2
GGVGSPYVSRL

SEQ ID NO: 86 - protein, 11 aa, Artificial Sequence
  ErbB3
GSLDHAHIVRL

SEQ ID NO: 87 - protein, 11 aa, Artificial Sequence
  ErbB4
GSMDHPHLVRL

SEQ ID NO: 88 - protein, 11 aa, Artificial Sequence
  Ret
GQVNHPHVIKL

SEQ ID NO: 89 - protein, 11 aa, Artificial Sequence
  Trk-NGFR
GMLQHQHIVRF

SEQ ID NO: 90 - protein, 11 aa, Artificial Sequence
  Trk-NGFR
GNLQHEHIVKF

SEQ ID NO: 91 - protein, 11 aa, Artificial Sequence
  Trk-NGFR
GDLQHRHIVRF

SEQ ID NO: 92 - protein, 11 aa, Artificial Sequence
  Trk-NGFR
GNLQHRHIVRF

SEQ ID NO: 93 - protein, 11 aa, Artificial Sequence
  Syk
GQLDNPYIVRM

SEQ ID NO: 94 - protein, 11 aa, Artificial Sequence
  Zap70
GQLDNPYIVRL

SEQ ID NO: 95 - protein, 11 aa, Artificial Sequence
  Jak1
GNLYHENIVKY

SEQ ID NO: 96 - protein, 11 aa, Artificial Sequence
  Jak2
GSLQHDNIVKY

SEQ ID NO: 97 - protein, 11 aa, Artificial Sequence
  Jak3
GALHSDFIVKY

SEQ ID NO: 98 - protein, 10 aa, Artificial Sequence
  IRK
GFTCHHVVRL

SEQ ID NO: 99 - protein, 10 aa, Artificial Sequence
  IRK
GFTSHHVVRL

SEQ ID NO: 100 - protein, 12 aa, Artificial Sequence
  IRK
GGFTCHHVVRLL

SEQ ID NO: 101 - protein, 12 aa, Artificial Sequence
  Irk
GFTCHHVVRRLL

SEQ ID NO: 102 - protein, 11 aa, Artificial Sequence
  Irk
GGFTCHHVVRL

SEQ ID NO: 103 - protein, 10 aa, Artificial Sequence
  Irk
GGFTCHHVVR

SEQ ID NO: 104 - protein, 11 aa, Artificial Sequence
  Irk
GFTCHHVVRLL

SEQ ID NO: 105 - protein, 9 aa, Artificial Sequence
  Irk
GFTCHHVVR

SEQ ID NO: 106 - protein, 8 aa, Artificial Sequence
  Irk
GFTCHHVV

SEQ ID NO: 107 - protein, 12 aa, Artificial Sequence
  Irk
GGGFTCHHVVRL

SEQ ID NO: 108 - protein, 11 aa, Artificial Sequence
  Irk Position 11 is benzoylated
GGFTCHHVVRK

SEQ ID NO: 109 - protein, 11 aa, Artificial Sequence
  Irk
GGFTSHHVVRL

SEQ ID NO: 110 - protein, 9 aa, Artificial Sequence
  Irk
GGFTCHHVV

SEQ ID NO: 111 - protein, 11 aa, Artificial Sequence
  Irk Position 5 is alanine-beta-amino-cysteine
GGFTCHHVVRL

SEQ ID NO: 112 - protein, 11 aa, Artificial Sequence
  Irk Position 5 is lysine-epsilon-amino-cysteine
GGFTCHHVVRL

SEQ ID NO: 113 - protein, 10 aa, Artificial Sequence
  Irk
GGFTHHVVRL

SEQ ID NO: 114 - protein, 11 aa, Artificial Sequence
  Alk2
GMLRHENILGF

SEQ ID NO: 115 - protein, 11 aa, Artificial Sequence
  Alk3
GLMRHENILGF

SEQ ID NO: 116 - protein, 11 aa, Artificial Sequence
  TrkB
GNLQHEHIVKF

SEQ ID NO: 117 - protein, 11 aa, Artificial Sequence
  DDR1
GRLKDPNIIRL

SEQ ID NO: 118 - protein, 11 aa, Artificial Sequence
  DDR2
GRLKDPNIIHL

SEQ ID NO: 119 - protein, 11 aa, Artificial Sequence
  Tyk2
GTLYHEHIIKY

SEQ ID NO: 120 - protein, 11 aa, Artificial Sequence
  Eph-B4
GQFEHPNIIRL

SEQ ID NO: 121 - protein, 11 aa, Artificial Sequence
  ITK/TSK
GKLSHPKLVQL

SEQ ID NO: 122 - protein, 11 aa, Artificial Sequence
  ACK
GSLDHRNLIRL